(19) United States
(12) Patent Application Publication
(10) Pub. No.: US 2026/0263147 A1
(43) Pub. Date: Sep. 10, 2026

(54) CATHETER MAGNETIC ACTUATION AND INSERTION CONTROL, CATHETER LOCALIZATION AND CONTACT FORCE ESTIMATION, AND/OR MAGNETICALLY ACTUATED CATHETER WITH STIFFENING ELEMENT TO CONTROL DEFLECTION

(71) Applicant: CASE WESTERN RESERVE UNIVERSITY, Cleveland, OH (US)

(72) Inventors: Murat Cenk Cavusoglu, Cleveland, OH (US); Mark Griswold, Cleveland, OH (US); Taoming Liu, Cleveland, OH (US); Nicole Seiberlich, Cleveland, OH (US); Dominique Franson, Cleveland, OH (US); Eser Erdem Tuna, Cleveland, OH (US); Nathaniel Lombard Poirot, Cleveland, OH (US)

(21) Appl. No.: 19/166,769

(22) PCT Filed: Mar. 20, 2024
(86) PCT No.: PCT/US2024/020664

§ 371 Date: Sep. 18, 2025

Related U.S. Application Data
(60) Provisional application No. 63/491,157, filed on Mar. 20, 2023.

(51) Int. Cl. A61B 18/14; A61B 90/00

(52) U.S. Cl. CPC A61B 18/1492; A61B 90/37; A61B 2090/374

(57) ABSTRACT

This disclosure relates to catheter magnetic actuation and insertion control, catheter localization (e.g., tracking) and contact force estimation, and/or magnetically actuated catheters with a variable stiff segment. The systems, devices and methods herein can include or be used in conjunction with interventional magnetic resonance imaging (MRI). A method includes determining an axial position of a distal portion of a device based on position data, in which the position data is representative of a spatial position of at least a flexible distal portion of the device, and the device includes at least one actuation coil at a distal portion of the device. The method also includes determining an actuation control signal to provide current to the at least one coil to deflect the distal portion of the device based on the position data and target data, in which the target data is representative of at least one of a location of a target site and a trajectory of the distal portion of the device to the target site.

### CROSS-REFERENCE TO RELATED APPLICATION

This application claims the benefit of priority to U.S. Provisional Patent Application No. 63/491,157, filed Mar. 20, 2023, which is incorporated herein by reference in its entirety.

### GOVERNMENT INTEREST

This invention was made with government support under IIP-1700839 and IIS-1563805 awarded by the National Science Foundation and HL153034, HL163991 and EB018108 awarded by the National Institute of Health. The government has certain rights in the invention.

### TECHNICAL FIELD

This disclosure relates to catheter magnetic actuation and insertion control, catheter localization and contact force estimation, and/or magnetically actuated catheter with a variable stiff segment.

### BACKGROUND

Physicians commonly perform device insertion procedures for diagnostic or interventional purposes, such as tissue biopsy, drug delivery or other surgical intervention. As one example, a needle catheter or other device is inserted in such a way that the needle tip intersects a target of interest (e.g., a tumor). As another example, a catheter can be inserted into the heart to deliver a therapy (e.g., ablation) to heart or brain tissue. These procedures are complicated by the fact that devices are subject to bending while inside the tissue and that the target may move, due either to physiological motions, such as breathing or heartbeat, or due to tissue deformations resulting from the device-tissue interaction.

### SUMMARY

This disclosure relates to catheter magnetic actuation and insertion control, catheter localization and contact force estimation, and/or magnetically actuated catheter with a variable stiff segment.

In one example, a system includes one or more non-transitory media storing data and executable instructions, in which the data comprises position data representative of a spatial position of at least a flexible distal portion of a device, and target data representative of at least one of a location of a target site and a trajectory of the distal portion of the device to the target site. A processor is configured to access the non-transitory media and execute the instructions to perform a method. The method includes determining an axial position of the distal portion of the device based on the position data. The method also includes determining an actuation control signal to provide current to at least one coil at the distal portion of the device to deflect the distal portion of the device based on the position data and the target data. The method also includes determining an insertion control signal to actuate an insertion actuator coupled to the device to move at least the distal portion of the device axially, in which the insertion control signal and the actuation control signal are coordinated to advance the distal portion of the device along the trajectory.

In another example, a method includes determining an axial position of a distal portion of a device based on position data, in which the position data is representative of a spatial position of at least a flexible distal portion of the device, and the device includes at least one actuation coil at a distal portion of the device. The method also includes determining an actuation control signal to provide current to the at least one coil to deflect the distal portion of the device based on the position data and target data, in which the target data is representative of at least one of a location of a target site and a trajectory of the distal portion of the device to the target site. The method also includes generating an insertion control signal to actuate an insertion actuator coupled to the device to move at least the distal portion of the device axially, in which the insertion control signal and the actuation control signal are coordinated to advance the distal portion of the device along the trajectory. The method can be computer implemented, such as based on one or more processors executing the instructions to perform the method.

In yet another example, a system includes one or more non-transitory media storing data and executable instructions, in which the data comprises device model data representing a mathematical model of a device and anatomical data representing anatomy of a patient's body. A processor is configured to access the memory and execute the instructions to perform a method. The method includes estimating a model-based estimate of shape and/or location of the device based on the device model data, the anatomical data and actuation inputs applied to control current to at least one coil at the distal portion of the device. The method also includes estimating an image-based estimate of a shape and/or location of the device based on image artifacts detected in one or more images, in which the image artifacts are representative of magnetic actuation of the at least one coil in response to the actuation inputs. The method also includes combining the model-based estimate and the image-based estimate to provide localization data representative of the location, orientation and/or shape of the device relative to the anatomy.

In yet another example, a method includes storing, in one or more non-transitory media, device model data representing a mathematical model of a device and anatomical data representing anatomy of a patient's body. The method also includes estimating a model-based estimate of shape and/or location of the device based on the device model data, the anatomical data and actuation inputs applied to control current to at least one coil at the distal portion of the device. The method also includes estimating an image-based estimate of a shape and/or location of the device based on image artifacts detected in one or more images, in which the image artifacts are representative of magnetic actuation of the at least one coil in response to the actuation inputs. The method also includes combining the model-based estimate and the image-based estimate to provide localization data representative of the location, orientation and/or shape of the device relative to the anatomy.

In still another example, a catheter includes an elongate catheter body extending between a proximal end portion and a distal end portion, in which the catheter body has a lumen extending longitudinally through the catheter body, and the distal end portion is flexible. A coil is disposed at the distal end portion of the catheter body and configured to deflect the distal end portion in response to a magnetic field. An elongated stiffening element extends between spaced apart proximal and distal ends thereof, in which the stiffening element is configured to be moveable axially along the length of the catheter body.

### BRIEF DESCRIPTION OF THE DRAWINGS

FIG. 1 depicts an example of a system environment.

FIG. 2 shows examples of images of a catheter at different levels of deflection.

FIG. 3 depicts an example set of standard contrast and positive contrast single-slice images of an example catheter at different deflection levels.

FIG. 4 shows images of the catheter at different time points in a trajectory.

FIGS. 5A and 5B show a comparison of different device position detection methods.

FIG. 6 depicts images demonstrating effects of synchronizing catheter actuation with MR imaging.

FIG. 7 shows examples of different timing schemes for catheter actuation and MR imaging.

FIG. 8 depicts an example of an intervention system.

FIGS. 9A and 9B depict an enlarged view of parts of an example catheter.

FIG. 10 is a block diagram showing an example of actuator control circuitry that can be implemented for driving one or more actuator coils.

FIG. 11 is a block diagram showing another example of actuator control circuitry that can be implemented for driving one or more actuator coils.

FIG. 12 is a block diagram showing an example of insertion control circuitry that can be implemented for controlling insertion of a catheter and/or stiffening element.

FIG. 13 is an example of an insertion apparatus that can be implemented to axially position a catheter and/or a stiffening element.

FIGS. 14A, 14B and 14C are conceptual diagrams showing different amounts of deflection of a distal portion of a catheter.

FIG. 15 is a flow diagram of an example method for device localization.

FIG. 16 is a flow diagram of an example method for coordinated actuation and insertion control.

### DETAILED DESCRIPTION

This disclosure relates to catheter magnetic actuation and insertion control, catheter localization (e.g., tracking) and contact force estimation, and/or magnetically actuated catheters with a variable stiff segment. The systems, devices and methods described herein can include or be used in conjunction with interventional magnetic resonance imaging (MRI).

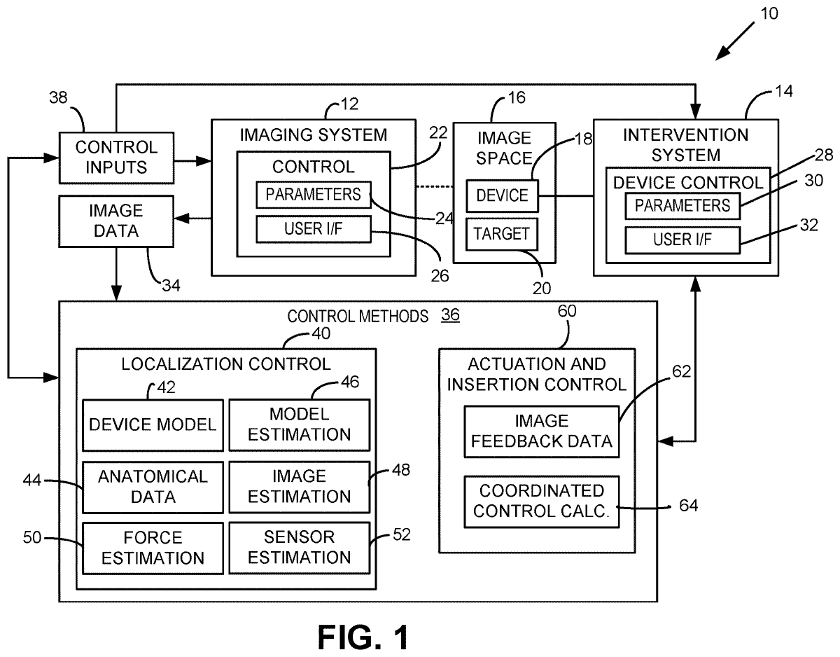

FIG. 1 depicts an example system environment 10, which can be configured to implement the various systems and methods described herein. The system environment 10 includes an imaging system 12 and an intervention system 14. The imaging system 12 can be external to the patient's body or internal to the patient's body and configured for intra-operative imaging of an image space 16. As used herein, intra-operative refers to utilizing the imaging during a procedure, such as a diagnostic procedure and/or an interventional procedure that is being performed with respect to the patient's body, which forms at least a portion of the image space 16. The image space 16 includes at least a portion of a patient's body and also includes one or more devices (e.g., one or more instruments moveable within the patient's body) 18 and a target 20. The target 20 is a desired location in the image space 16 to which the device 18 is being moved. The target 20, which can be fixed or moveable, further may be an intermediate point along a trajectory or a final destination on such trajectory. As used herein, a trajectory refers to a path of the device 18 through space (e.g., three-dimensional space). In some examples, the trajectory can be computed as an estimated path through one or more anatomical structures (e.g., vessels, cavities, organs and the like). Also, or as an alternative, the trajectory can be a vector that includes direction and magnitude components along a path through space.

By way of example, the imaging system 12 can be implemented according to various forms of intra-operative medical imaging modalities, including, magnetic resonance, computerized tomography, and ultrasound imaging (and other slice based intra-operative imaging modalities), as well as fluoroscopy (and other projection based intra-operative imaging modalities). The imaging system 12 provides image data 34 that corresponds to a time-based sequence of images of the image space 16, including the target 20 and the device located therein. Changes in the sequence of images provided by the image data 34, for example, can represent movement of the target 20, the device 18 as well as other matter (e.g., surrounding tissue) measurable by the imaging modality employed by the imaging system 12. The imaging data can be provided to a display to visualize the sequence of images acquired over time as well as be stored in memory (e.g., one or more machine readable storage media) for image analysis. The imaging system 12 can include image processing techniques to characterize tissue and other objects (e.g., including device 18 and target 20) in the image space 16, such that image data 34 can include processed images. For a given time step, the image data 34 can be a two-dimensional spatial visualization (e.g., an image across a plane) or three-dimensional spatial visualization (e.g. an image across a volume), and thus the image data can include pixels or voxels, accordingly. The analysis and processing of the images disclosed herein thus is with respect to the pixels or voxels in the image data 34.

The imaging system 12 includes a control system 22 to control imaging the image space 16 over time (e.g., over one or more imaging time steps). The control system 22 can control the imaging process over time according to a set of time variable input parameters 24. One or more of the input parameters 24, including activation and deactivation of the imaging system 12, can be provided in response to a user input via a user interface 26. The user interface 26 can be a human-machine interface to interact with the imaging system 12, such as to set one or more parameters 24 or otherwise configure the imaging system before or during an imaging procedure.

As mentioned, the intervention system 14 includes a device control system 28 to control the intervention system, which can be employed to implement a medical intervention with respect to a portion of the patient's body located in the imaging space 16. The control system 28 can control the intervention system 14, including controlling motion and/or positioning of the device(s) 18, over time according to a set of associated operating parameters 30. The control system 28 also includes a user interface 32 that can be utilized to provide or adjust one or more of the parameters 30 in response to a user input, including activation and deactivation of the intervention system 14. The user interface 32 can include a human-machine interface to interact with the intervention system 14, such as to set one or more of the parameters 30 or otherwise configure and/or control the intervention system 14 before and/or during an interventional procedure.

As described herein, the intervention system 14 is configured to perform a percutaneous intervention with respect to the patient's body. For example, the device 18 is a catheter configured to be inserted percutaneously into the patient's body and advanced to the target 20 for performing a respective procedure (e.g., diagnostic and/or treatment of tissue). The catheter 18 has an arrangement of one or more coils for magnetic actuation disposed along the distal end portion thereof configured to deflect the distal end portion responsive to applied current and/or a magnetic field. In addition to deflection of the distal end portion, the catheter can also be advanced and retracted inside the patient's body from its distal end. One or more of the coils can be multi-axis coils, such as described herein. In an example, the catheter a plurality of sets of coils at a plurality of spaced apart locations along a distal end portion of the catheter, such as one set adjacent the distal tip of the catheter and at least another set of coils spaced a distance axially apart from the catheter tip.

The control system 28 is configured to generate output signals to control the coil in the device 18 such as input electric current selectively provided to energize respective coils as to cause movement of the catheter toward a desired point on a given trajectory between the endpoint and the target site. The control system 28 can additionally generate output signals to control the axial insertion and retraction of the catheter. For example, the control system 28 includes actuation controls configured to implement the control methods programmed to control device positioning and actuation, such as including inverse kinematics and Jacobian controls to adjust the position of the catheter (or other instrument) toward the target 20 based on active control inputs. In response to the current applied by the intervention system 14, for example, magnetic moments can be generated on each respective coil including the tip or other location where the coils reside along the elongated body portion of the catheter. The magnetic moments generated at each coil sets interact with the magnetic field of the MR scanner implemented at the imaging system 12 to deflect the flexible elongated body of the catheter in a desired direction with respect to a central longitudinal axis thereof.

The catheter 18 also includes a lumen into which one or more tools can be inserted through (e.g., partially or completely through). In some examples, the tool is a stiffening element (e.g., also referred to as a rigid shaft) that is axially movable along the length of the catheter body. The stiffening element extends longitudinally between proximal and distal ends thereof, and can be straight or have one or more curved portions along its length. In one example, the distal end of the stiffening element can be inserted into the catheter lumen to terminate at a distal insertion point, such as within the flexible distal end portion of the catheter. In another example, the stiffening element can be inserted over and along the length of the catheter body to terminate at an axial position, such as circumscribing part of the distal end portion of the catheter. The presence of the stiffening element within the catheter lumen or along an exterior of the catheter body is configured to inhibit (e.g., prevent) bending of proximal portion of the catheter and control the amount of deflection that can be implemented by the distal portion of the catheter, such as described herein. The position of the flexible tool within the catheter can be fixed (e.g., by a locking mechanism). As an alternative, the elongated tool can be a flexible tool, such as a second flexible catheter, an injection needle, a puncture needle, guidewire, or other elongate flexible tool dimensioned and configured to be inserted within the catheter lumen according to needs of the respective procedure.

The systems and methods described herein thus can be configured to perform motion control of the catheter and the tool, which resides within the catheter according to the motion control of the catheter. As described herein, the motion control of the catheter and the flexible tool therein can include magnetic actuation of the flexible catheter tip and tool within the tip as well as insertion control thereof. In some examples, the intervention system 14 includes robotic actuation tools or instruments, which are inserted into the patient and guided to the target (e.g., a fixed or moving target) 20 based on localization via intra-operative medical imaging (e.g., via imaging system 12). As one example, the intervention system 14 can be implemented as a robotically controlled catheter ablation system in which the catheter tip (e.g., device 18) is to ablate target tissue 20 based on control parameters 30 that vary with respect to time.

In the example of FIG. 1, the control inputs 38 can provide active control for the imaging system 12 and/or the intervention system 14. The control inputs 38 set one or more of the image parameters 24 of the imaging system 12 determined to maximize information gain about the current state of the system 10. Additionally or alternatively, the control inputs 38 also can set one or more of the control parameters 30 of the intervention system 14 also to maximize information gain for the system 10. The control parameters 30 set via the control inputs 38 can be employed to steer and adjust the position of the device (e.g., catheter or needle) in substantially real time. The control inputs 38 to the intervention system 14 thus can be utilized to navigate a tip of the device to the target site 20 in the patient's body as well as to implement relative motion control (e.g., motion cancellation) once at the target site so that the device 18 moves commensurately with movement of the tissue target (e.g., to maintain continuous contact).

As disclosed herein, the system 10 includes one or more control methods 36. The control methods 36 can be implemented as machine-readable instructions stored in one or more non-transitory media, which are executable by one or more processors to perform the functions described herein. That is, the control methods can be implemented by a computer device, which has interfaces coupled to the control inputs 38, the source of the image data 34 and the intervention system 14. While the control methods 36 are shown outside of the intervention system 14, it is to be understood that the methods, in whole or in any part thereof, can be implemented within the intervention system 14.

In the example of FIG. 1, control methods 36 include localization control methods 40 and actuation and insertion control methods 60. The actuation and insertion control methods 60 are configured to implement coordinated control (also referred to herein as synchronized control) of insertion motion and magnetic actuation (e.g., navigation) of the device 18. As described herein, the device 18 can be a catheter or other device having a flexible (e.g., bendable or pliant) portion (e.g., distal end portion or tip) having one or more coils, each of which coils having one or multiple axes. Multi-axis coils help avoid magnetic actuation singularities.

In some examples, the device 18 is a catheter having a central lumen through which a second device (e.g., a second flexible catheter, injection needle, guidewire, puncture needle, or a stiff rod) can be inserted. The actuation and insertion control methods 60 can be configured to implement the coordinated insertion and magnetic actuation based on image feedback data 62 derived from the image data 34. For example, the image feedback data can be representative of anatomy and the relative location of the device 18. The actuation and insertion control methods 60 also includes a coordinated control calculator 64 programmed to compute parameters to implement the coordinated control between magnetic actuation and insertion actuation. The control calculator 64 can derive the control parameters based on the image feedback data, the control inputs and other parameters 30 of the device control 28, such as including a location of the distal end portion of the device 18, contact between the distal end portion and surrounding tissue. As used herein, the coordinated control (e.g., implemented by coordinated control calculator 64) refers to the motions controlled by the magnetic actuation (controlling the deflection/bending of the device) and the insertion actuation (controlling how far the catheter is inserted). For example, the coordinated controls are performed in a way such that the actuation/control of the motion of one depends on the performance and feedback of the motion of the other (and vice versa). In other words, the motions controlled by the two actuation schemes for deflection and insertion are synchronized (e.g., temporally, in magnitude and/or direction) in a way that enables adjusting of their motion based on the motion/path of the others.

As a further example, the control parameters for the magnetic actuation include the current applied to each of the coils and/or the rate of change in the current applied to the respective coils. The control parameters of the insertion actuation can include how much the device is inserted and/or speed of insertion. In one example, a single controller includes the actuation and insertion control methods 60 and is thus configured to compute the control parameters of both of the actuation and insertion systems, which controls the control parameters for both actuators together. In another example, separate controllers include each the respective actuation and insertion control methods 60 and compute respective control parameters based on monitoring the current state and progress of the other(s).

The control parameters can be calculated based on one or more of planning data, state information data, sensor data and/or estimated/predicted data. Planning data can include the planned trajectory of the catheter. The state information data can include information describing the position and/or orientation of the catheter tip or other points along the catheter (such as actuator coils). The state information can be determined by the imaging system or other independent sensors. The state information can also include the shape of the catheter, as determined by the imaging system and/or computational model of the catheter, internal state variables of the actuators (e.g., the current outputs of the amplifiers connected to the actuation coils, internal displacements of the insertion mechanism). The current and predicted state data further can include the current and predicted future locations of the anatomical target, measurements/estimates of the biological perturbations (e.g., blood flow velocity). The measured and computed information can be stored (e.g., in memory) for computing the control parameters, as described herein.

The localization control methods 40 are programmed to localize and estimate the shape and associated forces of the device 18. The localization control methods 40 can be implemented based on the image data 34, a device model 42 and anatomical data 44. For example, the device model 42 represents static and/or kinetic/motion properties of the device 18 such as by specifying a state transition probability density function for the device (e.g., catheter). The anatomical data 44 can be derived from pre-operative or real-time imaging (e.g., by imaging system 12). The localization control methods 40 can include a model estimation method 46 and an image estimation method 48. As described herein, the model estimation method 46 is programmed to provide a model-based estimate of device position (e.g., in two-or three-dimensional space) based on the model data and system data (e.g., device control parameters 30 and anatomical data 44). As an example, model estimation method 46 is programmed to determine a model-based estimate for the catheter employing a kinematic or mechanical model of the catheter that calculates the shape of the catheter from the applied actuation forces and torques, mechanical properties of the catheter, and the boundary conditions (e.g., entry point and direction of the catheter in the left atrium), as well as contact forces at the tip of the catheter (e.g., estimated or measured forces). Examples of some kinematic models that can be implemented by the model estimation method 46 include “Modeling and Validation of the Three Dimensional Deflection of an MRI-Compatible Magnetically-Actuated Steerable Catheter.” T. Liu, N. Lombard Poirot, D. Franson, N. Seiberlich, M. A. Griswold and M. C. Cavusoglu. In IEEE Transactions on Biomedical Engineering, Vol. 63, No. 10, October 2016, pp. 2142-2154; “Iterative Jacobian-Based Inverse Kinematics and Open-Loop Control of an MRI-Guided Magnetically-Actuated Steerable Catheter System.” T. Liu, R. Jackson, D. Franson, N. Lombard Poirot, R. K. Criss, N. Seiberlich, M. A. Griswold and M. C. Cavusoglu. In IEEE/ASME Transactions on Mechatronics, Vol. 22, No. 4, August 2017, pp. 1765-1776; “Jacobian-Based Task-Space Motion Planning for MRI-Actuated Continuum Robots.” T. Greigarn, N. Lombard Poirot, X. Xu, and M. C. Cavusoglu. In Robotics and Automation Letters, Vol. 4, No. 1, January 2019, pp. 145-152; “Analytical Computation of the Contact Force Jacobian for MRI-Actuated Robotic Catheter.” Y. Itsarachaiyot, R. Hao, and M. C. Cavusoglu. In Proceedings of the IEEE/RSJ International Conference on Intelligent Robots and Systems (IROS 2023), Detroit, MI, Oct. 1-5, 2023; and “Experimental Validation of the Pseudo-Rigid-Body Model of the MRI-Actuated Catheter.” T. Greigarn, R. Jackson, T. Liu, and M. C. Cavusoglu. In Proceedings of the IEEE International Conference on Robotics and Automation (ICRA 2017), Singapore, May 29-Jun. 3, 2017, pp. 3600-3605.

The image estimation method 48 is programmed to provide an image-based estimate of device position (e.g., in two-or three-dimensional space) based on the image data. For example, the image estimation method 48 can use image artifacts generated by coils/actuators for estimating catheter shape in two-or three-dimensional space. For example, the image estimation method 48 employs the position and/or orientation of the catheter tip and/or other points along the catheter (e.g., at actuator coils or separate localization coils on the catheter body). The position and/or orientation can be measured using a catheter localization/tracking algorithm (e.g., and MR-based catheter localization/tracking code, as described herein. The image estimation method 48 can interpolate the determined positions using a geometric and/or a mechanical model to estimate the shape of the rest of the catheter body.

The localization control methods 40 can also include a force estimation method 50 programmed to determine one or more forces at the device 18 within the image space 16. For example, the force estimation method 50 is programmed to determine an error between the model and image based estimates of the catheter shape and to estimate contact forces at the catheter tip based on the error. Additionally or alternatively, force estimation method 50 is programmed to determine an error between the model and image based estimates of the catheter shape to estimate forces distributed along the length of the catheter body, such as forces resulting from interactions between the catheter and the tissue and/or the blood flow.

The contact forces can be computationally estimated by a numerical algorithm such that the error between the model-based estimate of the catheter shape matches the image-based estimate of the catheter shape. For example, the numerical algorithm iteratively changes the contact force used in the model-based estimate of the catheter shape, until a measure of error, such as the Euclidean distance between the catheter tip position calculated from the model and the catheter tip position as estimated from the image based method, is minimized.

In some examples, the localization control methods 40 can include one or more sensor estimation methods 52 programmed to estimate shape and/or forces at the device based on signals provided by one or more sensors integrated at the distal end portion of the device 18. For example, one or more fiber Bragg grating (FBG) sensors are integrated (e.g., embedded) at the distal portion of the device, and the sensor estimation method 52 is programmed to estimate catheter (or other device) shape and/or forces based on measurements from the FBG. The sensor information can be combined with the other estimates, including model-based estimate and image-based estimates (provided by estimation methods 46 and 48).

By way of further example, the localization control methods 40 can provide the control inputs 38 to control the imaging system 12 to localize a tip (or other structural component) of the device 18 and the target 20. During such localization control, the intervention control 28 can employ its control input parameters 30, which can be derived from the actuation and insertion control methods 60, to control the device 18 for executing one or more interventional tasks. As disclosed herein, the interventional task can include directing the catheter towards the target via axial insertion of the instrument and/or to selectively activate one or more coils disposed along the length of the instrument, as disclosed herein, to deflect the distal end portion in a direction toward a desired trajectory to the target location.

Additionally or alternatively, the interventional task can include a combination of interventions, such as including applying a therapy to the target site. The target site may be fixed or moveable relative to the delivery mechanism located on the device 18 and the control input parameters can be utilized to implement movement of the distal end of the device to maintain contact at the target site (e.g., patient tissue). Examples of therapies that can be applied to one or more target tissue sites include ablation (e.g., RF ablation, cryoablation, microwave ablation, laser ablation, surgical ablation or the like), electrical stimulation of tissue (e.g., muscle tissue or nervous tissue), delivery of chemicals or other types of therapy that can be delivered percutaneously. One or more of the control parameters of the intervention control system 28 can be established by or be derived from the control inputs generated by the control methods 36, as disclosed herein, to implement each interventional task.

### Coupled Magnetic Actuation and Insertion Control

One example described herein includes systems and methods configured to provide for coupled magnetic actuation and insertion control for a magnetically actuated catheter. The following description of coupled magnetic actuation and insertion control provides a useful example of actuation and insertion control that can be used in any of the systems and methods described herein. Accordingly, reference can be made to other examples herein, such as including FIGS. 1 and 8-13 for additional context.

For example, the catheter includes a flexible tip that includes one or more coils, each of which coils has one or more axes. The systems and methods are configured to coordinate control of insertion motion in conjunction with magnetic navigation of the flexible tip responsive to actuation of the one or more coils (e.g., by a controller). In one example, the catheter tip includes a plurality of multi-axis coils disposed on or within the tip at respective locations. The axes of respective coils can be arranged to reduce (e.g., to compensate for, cancel or otherwise avoid) magnetic actuation singularities. The controller is configured to provide control signals to drive electronics of the catheter coil and insertion mechanisms. The drive electronics are further configured to control insertion (or withdrawal) of the catheter as well as bending of the catheter tip responsive to the control signals.

In a further example, the catheter is configured to be rotated about a central longitudinal axis. For instance, a base located at the proximal end includes a rotary drive mechanism (e.g., a voice coil, servo or other rotary actuator) that can be activated (e.g., manually or robotically) in response to a user input directing rotation of the catheter about its axis. For example, the controller includes a rotation control configured to provide a control signal to drive electronics, which are coupled to the rotary drive mechanism at the base of the catheter. The rotation control can thus control an amount of rotation of the rotary drive mechanism to rotate the catheter body about its central axis responsive to the user input.

In another example, the catheter includes a lumen into which another flexible tool can be inserted through (e.g., partially or completely through). The flexible tool can be a second flexible catheter, an injection needle, a puncture needle, guidewire, or other elongate flexible tool dimensioned and configured to be inserted within the catheter lumen. In another example, a stiff (e.g., rigid) shaft is inserted into the catheter lumen by an insertion length to limit bending of proximal portion and control the amount of deflection that can be implemented by the distal portion of the catheter, such as described herein. The position of the flexible tool within the catheter can be fixed (e.g., by a locking mechanism). The systems and methods herein can be configured to perform motion control of the flexible tool, which resides within the catheter according to the motion control of the catheter. As described herein, the motion control of the catheter and the flexible tool therein can include magnetic actuation of the flexible catheter tip and tool within the tip, insertion control of the catheter and tool. For example embodiments in which the catheter includes a rotary drive configured to implement rotation thereof, the motion control can also include rotation control of the catheter and flexible tool.

In some examples, the systems and methods can perform the motion control (e.g., magnetic actuation, insertion and rotation) under real-time MRI image feedback of the anatomy and the relative location of the catheter and/or the tool. In one example, the MRI image feedback can be used to implement such motion control under direct user control (e.g., manual control implemented based on the MRI images generated in real-time intraprocedurally). In another example, the MRI image feedback can be used to implement such motion control as autonomous robotic control to control respective drive mechanisms to reach a target site (e.g., specified by the physician on the provided MRI images). The MRI images can provide a reference view of the anatomy or be provided as real-time images. Additionally, the relative location of the catheter and/or the tool can be provided based on a mathematical model of the catheter shape or through direct measurement of the catheter and tool provided by the MRI imaging data.

As a further example, the MR image feedback is in the form of the position and/or orientation of the catheter tip or other points along the catheter (e.g., actuator coils or separate localization coils). The tip or other points can be as localized by the catheter localization algorithm, as described herein, and the location, orientation, and configuration of the anatomical target(s), as determined by anatomical imaging by the MRI. The feedback further can be used to provide the system with information on the current configuration of the catheter. The control system compares the current configuration of the catheter and the desired configuration (either provided by the physician directly through a user interface or calculated by the system based on the location of the anatomical target through a catheter trajectory planner). The error between the current and the desired configurations of the catheter is then used by the controller to correct the error and/or update the planned catheter motion trajectory.

### Catheter Localization, and Shape and Contact Force Estimation

As a further example, systems and methods configured to perform device localization, and shape and contact force estimation, such as can provide control feedback for coil actuation, catheter insertion/retraction, and/or imaging functions. The following description of catheter localization, and shape and contact force estimation provides a useful example of localization and shape and force estimation that can be used in any of the systems and methods described herein. Accordingly, reference can be made to other examples herein, such as including FIGS. 1 and 8-13, for additional context.

In the following, the device is described as a catheter; however, the device can include a catheter, guidewire, bioptome, ablation tool, and/or other tools, as described herein. As an example, a catheter includes a tube of a polymer (or other MRI-compatible material) with an arrangement of electrically conductive coils embedded on or in in a distal end portion thereof near the tip. The catheter can be steered by applying pre-determined combinations of current to the respective coils.

The localization and force estimation can be implemented based on a model of catheter using a Bayesian filter (such as, extended Kalman filter, unscented Kalman filter, particle filter) and one or more actuation inputs (e.g., provided by the motion controller). For example, anatomical information can be acquired from pre-operative and real-time medical imaging to provide geometry information for one or more anatomical structures of interest. The anatomical information can be used to identify entry and exit points as well as one or more trajectories through anatomical structures (e.g., vessels, cavities, organs and the like). As described herein, the systems and methods can be programmed to estimate catheter shape based on image artifacts created by the actuators. In some examples, the controller is configured to apply currents in the null-space of the actuation Jacobian so as to ensure creating image artifacts even for the cases where small or no actuation torque needs to be generated. In some examples, the systems and methods can store a dictionary or library (e.g., in memory) for a plurality of predicted artifacts. The localization function can include a search function programmed to search through the library (e.g., using non-iterative search techniques) to localize the actuation coils and identify its orientation based on the detected artifacts. In some examples, the localization function is programmed to determine an error between the model and image based estimates of the catheter shape and to estimate contact forces at the catheter tip based on the error. Additionally or alternatively, the localization function is programmed to determine an error between the model and image based estimates of the catheter shape to estimate distributed forces at the catheter tip and/or distributed along the length of the catheter.

In a further example, the catheter includes one or more fiber Bragg grating (FBG) sensors embedded on the catheter body (e.g., at the distal end portion and/or tip of the catheter). The controller can be coupled to the FBG sensors to receive respective FBG sensor signals representative of strain detected by the sensor. The controller can further include a catheter shape estimation function programmed to determine an indication of shape based on the FBG sensor signals. The indication of shape determined by the catheter shape estimation function can be combined with other location and shape information described herein to improve the accuracy of shape and orientation. In yet another example, the controller can further include a contact force estimation function programmed to determine an indication of contact force at the tip (e.g., at locations where sensors are positioned on the catheter) based on the FBG sensor signals. The contact force estimation function can provide a value representative of a normal force or three-dimensional contact force at the catheter tip.

As a further example, a catheter tracking system includes one or more tracking navigators, which enable tracking of image features at a higher frame rate than the image is updated. Navigators can also determine coordinates of image features, including those corresponding to devices. The systems and methods herein can be configured to use positive contrast navigators to actively detect the catheter prototype. The catheter tracking can also be interleaved with real-time background imaging (e.g., interleaved with standard contrast imaging).

Positive contrast can be implemented by scaling the moment of the slice-select refocusing gradient. The moment could be scaled as a percentage of the moment that would be needed to refocus the slice-select gradient in a standard-contrast acquisition. The scaling factor can be adjustable. A second dephasing gradient can be added along the third axis, such that for each navigator, the signal is dephased along both of the non-navigator axes. For the navigator along the slice-select direction, slice selection can be performed with normal gradients, and dephasing gradients for positive contrast can be applied to the other two axes.

A navigator block of multiple (e.g., three) orthogonal navigators could be implemented at user-defined intervals. The navigator block can be invoked after acquiring an entire background image, or after acquiring some of the k-space lines for the background image for a higher detection frame rate. As one example, the navigators were most commonly acquired after every 4/16 image k-space lines; for every image dataset, four navigator blocks were acquired. By playing the navigators more frequently reduces the effective frame rate of the background imaging.

From the Fourier slice theorem, given a line of data passing through the origin at a known angle in a 2D k-space dataset, a 1D inverse Fourier transform of that line of data is equivalent to the 2D Radon transform of the object signal at the same known angle. In terms of 2D spatial encoding, this is equivalent to acquiring a line of frequency-encoded data without applying phase encoding in the orthogonal direction; the data are spatially localized only along the direction of acquisition. The Fourier slice theorem can be leveraged to obtain rapid, 1D navigator profiles of the object. Instead of acquiring enough lines of k-space to reconstruct an image, a few lines of k-space at different angles are acquired. After performing a 1D inverse Fourier transform along each line, signal profiles can be generated to estimate the device position.

Navigators further can be added to the real-time imaging sequence with three 1D acquisitions along the slice-select, frequency-encoding, and phase-encoding directions. A slice-selective excitation was used to excite the detection area, and then a frequency-encoding gradient was played out along the navigator axis. The slice thickness for device detection could be set independently from the imaging slice thickness.

For two-dimensional (2D) detection, two orthogonal navigators passing through the center of k-space can be collected, and a 1D inverse Fourier transform performed to generate orthogonal signal profiles. The coordinates of the intersection of the maximum peaks in the signal profiles can be taken as representative of the position of the catheter. For 3D detection, a third orthogonal line of k-space can be acquired, and the same processing can be performed. More than 3 lines of k-space data can also be acquired, and the most likely location of the catheter position can be estimated using a method, such as, least squares optimization or maximum likelihood estimation, that would give the three dimensional coordinates of the point that would best fit the maximum peaks along each of the lines as the estimated catheter position. For example, at each deflection level, data are acquired at a range of positive contrast scaling factors. The scaling factor that visually resulted in the best detection can be selected empirically. The accuracy of detection can be determined by comparing the detected position with the coil artifact and catheter shaft that is visible in one or more images. Catheter detection and imaging are both performed in the scanner's gradient coordinate system (slice-select, frequency-encoding, phase-encoding axes), so the MR-detected catheter position can be overlaid onto the image without any coordinate transformations. In other examples, in which catheter detection and imaging are performed in different coordinate systems, image transformations can be used to co-register the detected catheter position and the image for visualization.

In the examples above, the imaging and detection were performed on energized catheters, resulting in background images with signal void artifacts surrounding the coils. The artifacts might obscure anatomy near the catheter tip when imaging in vivo. In order to remove the artifact from the background images, synchronization between catheter actuation and MR data collection can be used to permit image data collection when the current is off. Synchronization can be achieved using the scanner optical trigger. The actuation current remained on during navigator data acquisition.

The frequency of the current off intervals can be controlled from the MR scanner side, in which the trigger value is set to “high” at the beginning of each imaging block. The duration of the off intervals can be set by the catheter controller, in which the number of image k-space lines per imaging block and the repetition time (TR) of the sequence is used to calculate the duration. A catheter actuation “control pause” can also be added to the beginning of the navigator block in the imaging sequence. During this period, no MR data were collected, and the catheter actuated freely. As an example, the pause period can be set to one TR, such that image data were acquired for four TRs (current off), and then the catheter was energized for four TRs (three navigators, one TR pause).

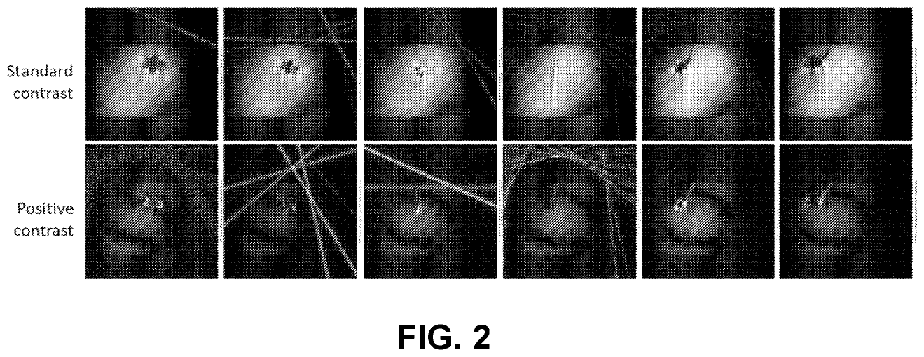

As a further example, a positive contrast navigator can be used to detect a static catheter. FIG. 2 shows examples of images of a catheter at different levels of deflection. The left-and right-most panels show large levels of deflection, corresponding to large positive and negative current values on the catheter coil, while the fourth panel from the left shows the catheter in a neutral position with no current applied. A larger artifact is observed with more current/higher levels of deflection. The top row of images shows standard contrast images, where the artifacts appear as signal voids. The bottom row of images shows positive contrast images of the catheter at the same positions, and the artifacts appear bright on a dark background. In the positive contrast images, the background signal is not completely spoiled. These images were acquired with the slice-select refocusing gradient removed, but without any additional spoiling. In other examples, dephasing gradients can be used along two axes, and adjustable-moment spoiler gradients. The full image is not needed for the navigator detection, but the images are helpful to visually understand the positive contrast patterns created by the energized catheter coils.

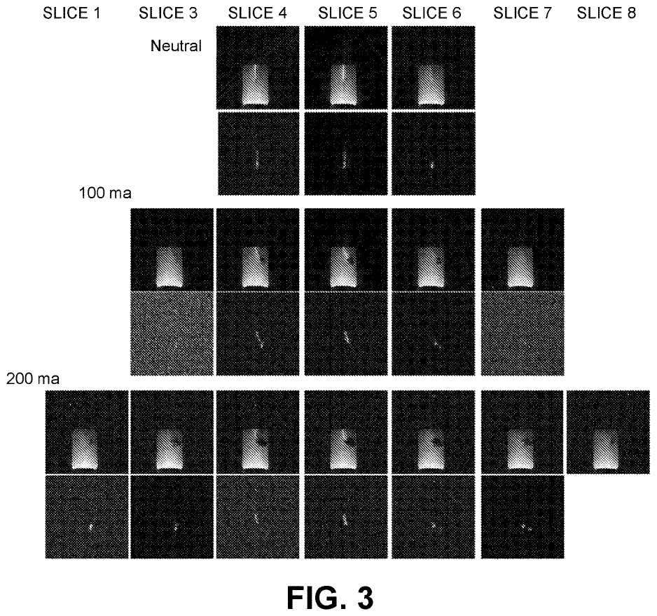

FIG. 3 depicts an example set of standard contrast and positive contrast single-slice images of an example catheter at different deflection levels, in which the catheter device is mounted at the top of the tank (top side of images), and positive and negative currents are applied to achieve left and right defections. The streaking artifacts in some images is due to interference. The example of FIG. 3 shows standard and positive contrast images of an example catheter at three deflection levels (e.g., neutral, moderate deflection at 100 mA, stronger deflection at 200 mA). For example, the image data were acquired with a multi-slice acquisition, and detection was performed at each slice independently, as described herein. Scan parameters for image acquisition were as follows: FOV=280×280 mm2; slice thickness=5 mm; TR/TE=3.53/1.61 ms; flip angle=12°.

At each position, the catheter is detected using the positive contrast navigators, and the detected position is overlaid onto the images. Data were acquired using a multi-slice sequence, and detection was performed independently at each slice. In the standard contrast images (e.g., in the bottom row of each image set), it can be seen that the artifacts from the energized coil extend over multiple slices, and the artifact volume is larger when more current is applied. For example, in FIG. 3, slice 5 is centered over the catheter shaft. Detection in slice 5 at both the 100 mA and 200 mA conditions locates the catheter proximal to the coil. However, detection visually improves in the neighboring slices. At 100 mA, detection appears centered along the long axis in slices 3 and 4, while at 200 mA, detection appears more centered in slices 2 and 6, and misses the catheter in slices 3 and 4. The positive contrast images thus are helpful in understanding the variability in detection by showing where the refocusing field produces positive contrast with high contrast-to-noise ratio (CNR).

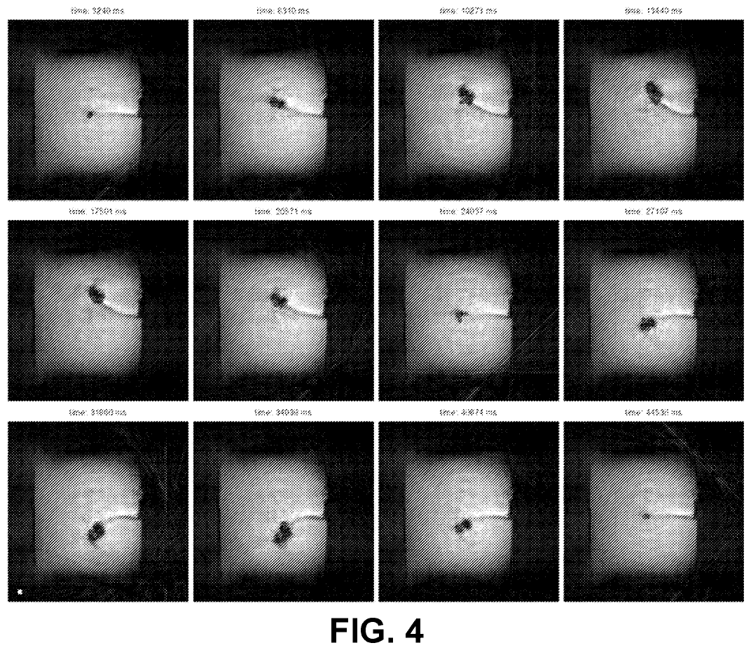

FIG. 4 shows images of the catheter at different time points in the trajectory reconstructed with through-time radial GRAPPA. For example, imaging can be performed with the following parameters: field-of-view=300×300mm²; matrix size=128×128; in-plane resolution=2.34×2.34mm²; slice thickness=5 mm; flip angle=5°; TR/TE=3.66/2.35 ms; bandwidth=795 Hz/pixel; number of coils=30. In FIG. 4, the predicted positions are shown along with an overlay of the coil position detected using MR navigators and an overlay of a camera system. The white box in the bottom left frame is given as a reference scale. An acceleration factor of 6 (24 of 144 radial projections) was tested. The data acquisition time for a single image and set of navigators was 99 ms. Reconstruction can be performed on-line or off-line with a through-time GRAPPA reconstruction using 60 measurements of calibration data.

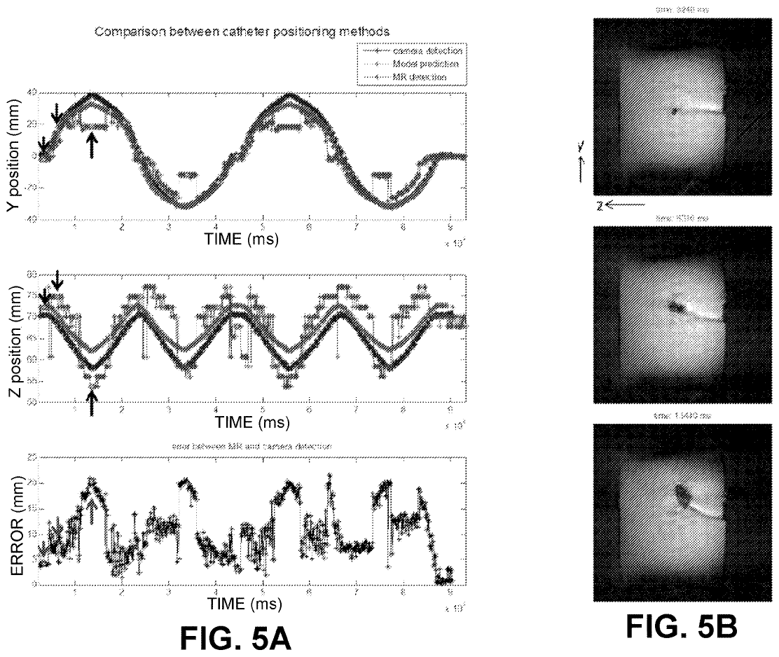

FIGS. 5A and 5B show a comparison of the two device position detection methods (MR and camera detection methods) and model prediction over time. The mean error between the MR and camera detection is shown in the bottom plot (e.g., as 10.5 mm (±5.3 mm)). The y axes of the top two plots show the position of the catheter coil relative to the base of the catheter, which is clamped by a plastic support structure at the top of the phantom. In the neutral position, the coil is in line with the catheter base in the y direction, and about 70 mm away from the base in the z direction. The corresponding times on the position plots are marked with arrows. As shown, the largest error occurs at peak deflections. For example, at deflections greater than 30 mm from neutral, the mean error was 15.2 mm (±4.4 mm). At deflections less than 30 mm, the error was 8.1 mm (±3.8 mm).

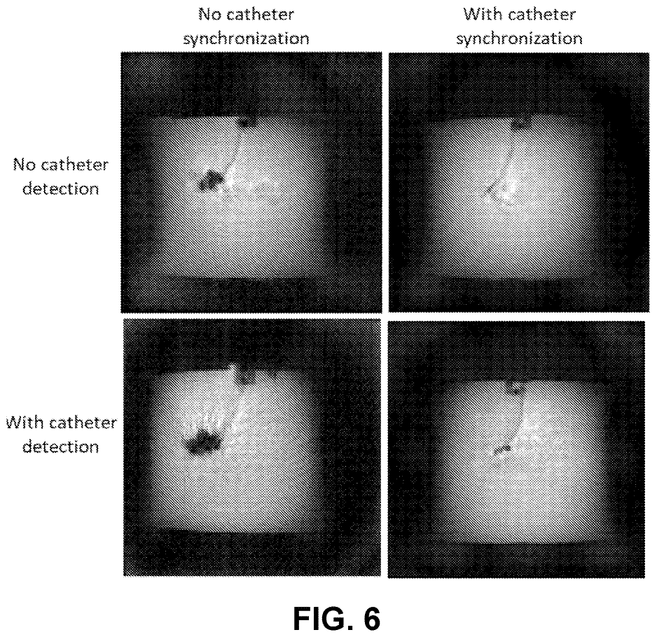

FIG. 6 depicts images demonstrating the effects of synchronizing catheter actuation with MR imaging, such as described herein by implementing interleaved catheter detection and artifact-free background imaging. Without synchronization, an artifact surrounding the coil is visible in the images (top left image). When the current is paused during image data acquisition, the artifact is removed and only the polymer catheter tube is visible (top right image). The synchronization is also compatible with the navigator-based catheter detection (bottom right image). In the example of FIG. 6, the images are from different scans, and the level of catheter deflection is visually matched between the panels but is not exact.

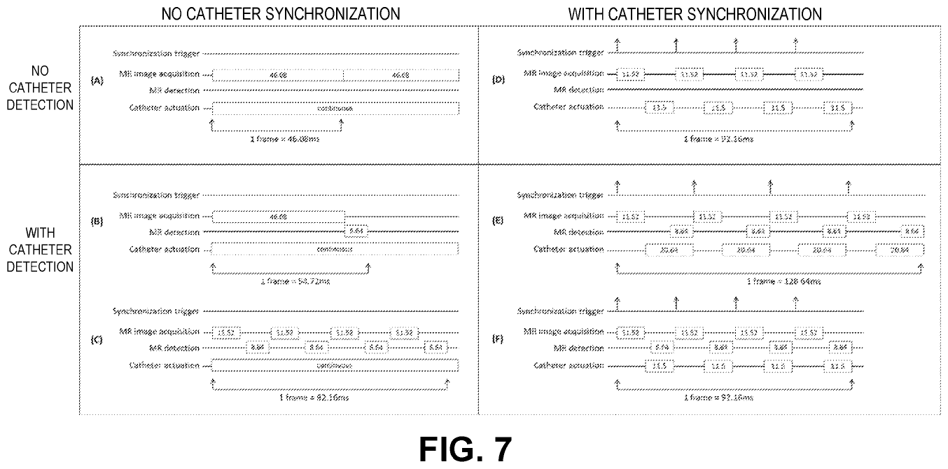

FIG. 7 shows example timing schemes (conditions A, B, D and E) used for each of the imaging conditions to generate the images in FIG. 6. Imaging conditions C and Fare other timing options when catheter detection is included, and were also tested in some experiments. In imaging condition C, image data acquisition is broken into 4 intervals per frame, and detection is performed more frequently than in condition B. In imaging condition F, the catheter on/off duty cycle is 50/50 and the total frame duration decreases compared to E, where the catheter is energized for longer in each “on” interval.

The relative timings and frequency of the detection, background imaging, and catheter actuation affect the total imaging frame rate and the detection rate. For example, if the catheter freely actuates without synchronization (top left panel, condition A), the imaging frame rate is 46.08 ms/frame. When device detection is added (bottom left panel, condition B), the image frame rate drops to 54.72 ms/frame, and the device position is updated once per frame. When actuation/background synchronization is introduced the image frame rate is 92.16 ms/frame (top right panel, condition D). When actuation, background imaging, and detection are interleaved, the frame rate is 128.64 ms/frame (bottom right panel, condition E). In this case, a control pause of 12 ms was used at the beginning of each navigator block.

While the timings in conditions B and E of FIG. 7 correspond to the examples shown in FIG. 6, it is also possible to adjust the interleaving patterns for more frequent detection and/or faster frame rates. Device detection can be interleaved after every four lines of imaging data (bottom left panel, condition C). In this example, the image frame rate drops to 92.16 ms/frame, but the device position is updated every 20.16 ms. The frame rate remains 92.16 ms/frame when actuation/background synchronization is added (bottom right panel, condition F) since the detection is performed during the catheter actuation intervals.

In the foregoing description, systems and methods use interleaved positive contrast detection navigators programmed to detect the position (e.g., localize) an MR-actuated catheter based on imaging data representative of a real-time imaging sequence (e.g., MRI image data). A flexible sequence was developed that combined the under-sampled radial acquisition with navigator blocks that can be implemented at user-programmable time intervals. The imaging sequence also can be synchronized with catheter actuation to achieve artifact-free background imaging with overlaid device detection.

Positive contrast was implemented by scaling the moment of the slice-select refocusing gradient. Full FOV images (see, e.g., FIGS. 2 and 3) show a conspicuous device signal surrounding the catheter coil and along the energized catheter shaft. This conspicuity may be acceptable if full FOV images are used for guidance. In this example, the device position can be updated at the same rate as imaging, and the background anatomy would have lower SNR compared to standard contrast imaging. The position detection followed the shape of 2D back-and-forth and 3D rectangular trajectories.

A changing actuation current for respective coils resulted in a refocusing field for positive contrast that varied over the trajectory, which could move the position of the maximum navigator signal relative to the coil. For instance, the error changed with the deflection level, and was highest with the most deflection. In one set of examples, a single detection current yielding the best detection in static catheter experiments was selected. Using the same synchronization trigger that used for artifact-free background imaging, the current applied to the catheter was cycled between a fixed detection current, the trajectory-defined current, and no current for MR-detection, catheter actuation, and artifact-free background imaging, respectively.

In some examples, the actuation coils can be simultaneously used for detection and to enable complex catheter motion. In other examples, separate detection coils can be adapted to generate refocusing fields that are localized around the coils to enable desired contrast detection for localization, and such coils can be used together with catheter actuation coils. Such detection coils can be used in combination with positive contrast imaging navigators to more systematically achieve high CNR, enhance coil detection coils, and reduce the empirical nature of tuning the positive contrast scaling factor.

Additionally, for some coil designs, the systems and methods can include a background subtraction function configured to subtract the background signal from the navigators before detection to help achieve higher coil CNR. The background subtraction can enhance the positive contrast scaling factor and sufficiently dephase background signal from the medium (e.g., in vivo) where the device is located. For example, the edges of the t close to the receiver coils sometimes generated stronger signal peaks than the catheter coil in the detection navigators. One way the background signal was measured was by taking the time-averaged signal from navigators acquired when the catheter was not energized at the beginning or end of the experiment. A second test used synchronization with the catheter control system to turn the catheter current off in alternating navigator blocks. In this way, the background signal could be updated throughout the scan. The background subtraction function can be used in combination with higher CNR detection coils.

The positive contrast navigators were implemented in the gradient coordinate system of the scanner, which puts the detected positions in the same coordinate system as the image data. One of the next steps of this project will be to export the background anatomical images and device positions to the catheter control system for real-time, closed-loop control of the catheter. As part of this step, all of the data will need to be transformed to the same coordinate system. Initial work has been done toward establishing a registration method.

Additionally, device detection, background imaging, and catheter actuation can be interleaved in one imaging sequence using a synchronization trigger. Without synchronization, a large artifact surrounds the catheter coil that could obscure the anatomy near the catheter tip, which is likely the anatomy of interest in a procedure. The on/off intervals and duty cycles can be tuned to minimum values needed to avoid displaying jittery catheter motion.

Rapid 2D and 3D detection of a catheter prototype mounted with actively powered coils is possible using positive contrast navigators. Detection navigators along three axes can be acquired with a desired spatial resolution and field-of-view per navigator block. The navigator block can be interleaved with standard-contrast background imaging at user-selected intervals. MR image data acquisition can be synchronized with catheter actuation for artifact-free background imaging.

### Magnetically Actuated Catheter Having a Stiff Element with a Variable Insertion Length

An elongated rigid shaft (e.g., stiffening element) is moveable axially along the length of a flexible catheter to inhibit bending and/or deflection of the catheter body based on the relative position of the shaft along the length of the catheter. In one example, the rigid shaft can be sized and configured for axial movement within a lumen of the body of the flexible catheter. In another example, the rigid shaft is configured as a rigid tube including a lumen, in which the lumen of the shaft has an inner diameter that is sized (e.g., slightly larger than) the outer diameter of the catheter body and the shaft is configured for axial movement over the catheter body.

The rigidity of the shaft is sufficient to inhibit bending of the distal end portion of the catheter responsive to magnetic actuation thereof while the shaft resides within the distal end portion. The following description of a catheter and stiffening element having a variable insertion length provides a useful example that can be used in any of the systems and methods described herein. Accordingly, reference can be made to other examples herein, including FIGS. 1 and 8-14, for additional context.

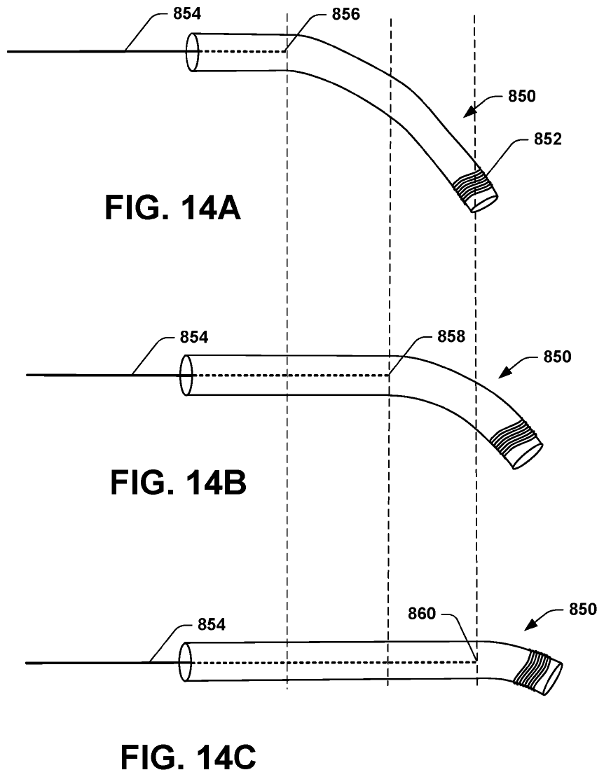

As one example, the catheter has a flexible tubular body portion that includes one or more, single or multi-axis embedded micro-coils coupled to or integrated within a distal end portion of the tubular body. As described herein, the flexible catheter, is thus configured for magnetic actuation of bending of the flexible catheter's distal end portion responsive to current in the respective coils. The rigid shaft, which is configured for insertion within the lumen of the flexible catheter, can be straight or curved. The inserted rigid shaft can have a distal end that can be positioned at desired position within the catheter lumen spaced proximally with respect to a distal end of the catheter. The rigid shaft is thus adapted to prevent the bending of the proximal portion of the flexible catheter and to provide a fixed shape for the portion of the catheter in which the shaft is located. A distal portion of the flexible catheter that extends beyond the distal end of the shaft remains flexible and further can be moved responsive to actuation of the coils thereof. The shaft also is configured to provide mechanical support for a proximal portion of the flexible catheter. Additionally, changing the insertion length of the rigid segment allows the point after which the distal portion of the catheter is allowed to bend to be changed. That is, the amount of deflection that can be implemented can be controlled (e.g., limited or enhanced) by controlling the insertion length of the rigid shaft within the distal portion of the catheter (see, e.g., FIG. 14).

Additionally, the rigid shaft can be inserted/retracted within the catheter lumen. Additionally, or alternatively, the shaft can be rotated axially within the catheter lumen. Thus, rotation of the rigid shaft in coordination with a change of its insertion length can be used to change the shape of the proximal segment of the flexible catheter. In some examples, the flexible distal segment of the catheter can be bent through magnetic actuation of the coils while keeping the rigid segment stationary. In other examples, the flexible catheter can be inserted/retracted while keeping the rigid segment stationary. As a result, the rigid shaft allows the catheter to advance or retract in tissue along a trajectory that is controlled by the bending of the distal flexible portion of the catheter.

Simultaneous insertion of the rigid shaft and the flexible catheter allows the catheter to be more easily inserted into the tissue by providing mechanical support along a trajectory determined by the rigid shaft (straight or curved) and bending of the distal flexible segment of the catheter.

The systems and methods disclosed herein thus can be applied to various forms of percutaneous procedures, such as tissue biopsy, therapy delivery and catheter-based procedures (e.g., cardiac ablation). For instance, the instrument system further can be a robotically controlled catheter that is actively controlled according to control parameters determined from localization of the target tissue and the instrument configuration, such as disclosed herein.

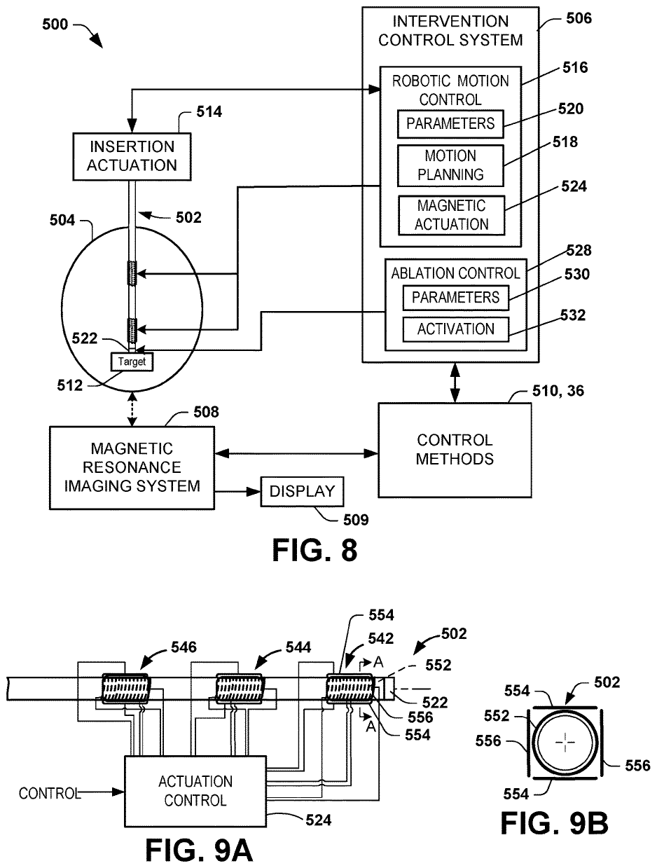

As a further example, FIG. 8 depicts an example of an interventional system 500 representative of delivery of ablation therapy via a catheter 502 that is insertable percutaneously into a patient's body, demonstrated schematically at 504. This system 500 includes an intervention control system 506 that is configured to implement one or more interventional control functions to operate one or more devices for diagnosing and/or treating the patient's body 504. The intervention control system 506 is a useful example of the control system 28 shown in FIG. 1. The system 500 also includes an imaging system demonstrated in this example as a magnetic resonance imaging (MRI) system (also referred to as an imaging system) 508; although, other imaging modalities can be used in other examples.

The system 500 also can include one or more control methods 510, which can be implemented according to control methods described herein (e.g., the control methods 36 of FIG. 1, as well as described elsewhere herein). Thus, the control methods 510 can include a catheter localization method, such as described herein (e.g., control methods 40). Additionally, or alternatively, the control methods 510 can include an actuation and insertion control method, such as described herein (e.g., control methods 60). As another additional or alternative example, the catheter 502 can be implemented as a hybrid magnetically actuated flexible catheter that includes variable length insertion stiffening element adapted for insertion within a lumen of the catheter, as also described herein.

For example, the control methods 510 can be configured to provide control inputs to the imaging system 508 and the invention control system 506. The control methods 510 can be implemented separately from or as part of one or both of the imaging system 508 and/or intervention control system 506. The control methods 510 can be implemented as machine instructions (executable by one or more processors) to generate one or more control inputs in response to imaging data acquired by the imaging system 508. The control methods 510 can implement localization and control (e.g., method 36 or 60) for the imaging system 508 and the intervention control system 506.

The imaging system 508 can acquire image data (e.g., 3D image data) of the patient's body including the catheter 502 and the corresponding target site 512. For example, the imaging system 508 is configured to image the catheter tip (of device 18) and the target tissue (e.g., tissue 20), such as on the pulmonary vein or other tissue structure and provide corresponding image data in substantially real-time (e.g., generate a new set of image data about every 20 ms to 50 ms). The image data can be preprocessed by the imaging system 508 and utilized by the control methods 510 to provide corresponding input control parameters to the MRI system 508 for active sensing of the target 512 and catheter 502. Additionally, the control methods 510 can provide corresponding input control parameters to the intervention control system 506 for controlling intervention with respect to the patient's body 504. The imaging system 508 can further generate an output visualization to provide a real-time display 509 according to the image being acquired.

In the following example, the intervention is provided via the catheter 502 that is inserted percutaneously. However, it is understood that the intervention system 500 can implement a variety of other diagnostic and/or treatment devices, which may be inserted internally within the body 504 or be provided externally (e.g., sources of radiation) applied to the internal target 512 that is actively localized.

In the example of FIG. 8, the intervention control system 506 includes a robotic motion control 516 to control operation of an arrangement of motion components (e.g., motors, actuators, coils or the like) to position the catheter 502 at a desired position relative to the patient's body. For example, the robotic motion control 516 can include a motion planning module 518 to determine control parameters 520 for adjusting the position and/or orientation of the catheter 502 in a coordinate system of the patient and imaging system 508. The robotic motion control 516 can be fully automated in response to the control parameters 520 provided by motion planning 518 and the control methods 510. In other examples, the robotic motion control can be co-robotically controlled, meaning that the parameters 520 can be modified based on user interaction and resulting catheter positioning is robotically assisted.

In the example of FIG. 8, the system 500 includes an axial actuation component 514 that can be utilized to adjust the axial position of a distal end of the catheter 502 in response to the control signals provided by robotic motion control 516. For example, the motion planning module 518 can determine control parameters 520 for axially adjusting the position and/or orientation of the catheter 502 in a coordinate system of the patient and imaging system 508. The axial actuation 514 can include a linear actuator (e.g., linear motor or servo motor) configured to advance the catheter body generally in an axial direction along a trajectory to position the catheter tip 522 into contact with the target 512.

In addition to adjusting the position of the catheter axially along the path via the actuation 514, the intervention control system 506 can include magnetic actuation control 524 to provide for translational motion with respect to three orthogonal axes in three-dimensional space. For example, the catheter magnetic actuation control 524 controls current applied to coils attached to the catheter body to affect deflection of the catheter in two orthogonal axes relative to its longitudinal axis. For example, the catheter body can include a polymer tubing that is embedded with multiple sets of corresponding current carrying coils.

The catheter 502 can be steered in a couple of different manners. First of all, the magnetic field of the MRI system 508 can be used to directly actuate the catheter. Alternatively or additionally, a small robot (e.g., placed near the patient's knees) can move the catheter 502 axially, rotate, and bend. The catheter itself would have coils embedded to help identify its location and orientation from the image data.

As a further example, the catheter can be implemented as an MR-actuated steerable catheter (see, e.g., FIG. 9A) that can cause deflection along the catheter body along transverse to the longitudinal axis thereof in response to current supplied by the robotic motion control 516. For example, current through the coils generates magnetic torque when subjected to the magnetic field provided by the MRI system 508. The currents can be controlled remotely via the magnetic actuation control 524 automatically or in response to a user input to steer the catheter in conjunction with the axial actuation 514 to move the distal end 522 of the catheter along a given trajectory, such as to contact the target site. The catheter motion can be achieved, for example, by interlacing the imaging and catheter control signals in the MRI system.

As the moment applied by the actuation 514 is dependent on the relative orientation of the actuator coils and the magnetic field vector of the MRI system 508, the external loading on the catheter changes with deflection. Therefore, the robotic motion control 516 is configured to calculate forward kinematics (catheter deflection for a given set of actuator currents) requires the solution of a system of nonlinear equations. The robotic motion control 516 also computes corresponding inverse kinematics because of the complexity of the kinematics. In the given formulation, it would be possible to derive analytic expressions for the Jacobian. That is, Jacobian-based methods can be effectively used for inverse kinematics calculations. For example, the robotic motion control 516 of intervention system 506 can employ the damped least squares method, which avoids stability problems in the neighborhoods of singularities.

In an example, where the rigid shaft is used (e.g., for axial movement within or over the catheter body), the proximal part of the catheter has a fixed shape due to the rigid shaft, and as a result would not bend. The forward kinematics, the inverse kinematics and the Jacobian are calculated by taking the pose of the rigid shaft into account with respect to the length of the catheter body. The control algorithm is further configured to determine how the rigid insert needs to be inserted along with the regular catheter insertion and the coil actuation, based on the desired motion of the flexible portion of the catheter.

As described herein, the location of the catheter tip 522 and the target tissue 512 are simultaneously measured by the intra-operative MRI system 508 in real-time to implement the localization and controls. For instance, the control methods 510 can implement an active sensing scheme to control the intra-operative imaging system 508, optimally selecting position and orientation of the image slices to be acquired as well as other imaging parameters, for optimal localization of the target and the catheter. The motion planning 518 and actuation control 524 of the robotic motion control 516 can utilize the target and catheter location information extracted from the images acquired by the intra-operative MRI system as well as active control inputs selected for each time step to actively control the position and/or shape catheter tip to track the target tissue.

Once the distal end of the catheter 522, which can include an ablation delivery structure (e.g., one or more electrodes or other structures adapted for ablation delivery), is in contact with the target site 512, the ablation control 528 can deliver ablated therapy to the target. The ablation control 528 can operate based upon a set of ablation parameters 530 and an activation function 532 to supply the ablative therapy via the delivery mechanism (e.g., at the distal end 522) according to the parameters 530. In one example, the ablation control 528 can supply RF energy to the electrode at the distal end 522 of the catheter 502 and activate the RF energy to ablate the tissue at the contact site of the target 512 in response to the parameters 530 specifying amplitude, frequency, duration (e.g., duty cycle) of energy. In other examples, the ablation delivery structure can be configured to deliver one or more other forms of ablation, such as cryoablation, laser ablation, chemical ablation, etc. In an example, the catheter is an ablation catheter adapted to perform brain/cerebral catheter ablations, which can be implemented during interventional MRI of the brain. During application of the therapy or other intervention, the intervention control system 506, including robotic motion control 516 and ablation control 528, further can adjust the position of the distal end of the catheter across the target 512 to achieve a sufficient amount of ablation on the target site. For example, the distal end of the catheter can be moved in a sequence of patterns and ablate the tissue at each of a plurality of locations across the surface or through the volume of the target tissue. The sequence of patterns can be automated or responsive to user controls.

In order to achieve successful ablation, the catheter tip 522 needs to remain in constant contact with the tissue when the activation component 532 applies ablative therapy. Such contact should be maintained and follow the trajectory specified by the operator, despite disturbances from blood flow and tissue motions, as mentioned above. Accordingly, in some examples, the robotic motion control 516 dynamically corrects the position of the catheter to compensate for (e.g., cancel) the cardiac motions and/or perturbations resulting from blood flow, and/or other physiological effects. For instance, the robotic motion control 516 can employ active relative motion cancellation methods. The motion control 516 actively tracks the motion of the deformable tissue, so as to dynamically adjust the position of the tip of the catheter to remain stationary relative to the tissue. Once the tip of the catheter is “stationary,” the motion planning can be performed with respect to a stationary tissue frame, and the resulting catheter motion can be added to the active tracking motion.

To reduce the computational complexity associated with such active tracking, the motion control take advantage of the quasi-periodic nature of the target tissue motion (e.g., wall motion synchronized with the patient's heart beat), and the perturbations on the catheter motion due to non-steady blood flow. The motion control thus can employ generalized adaptive filters for estimating the upcoming motion of the target tissue and the catheter. Once estimates of the upcoming catheter shape and target motions are available, even if they are not perfect, these estimates can be used to reduce the uncertainties in system dynamics. Reduced uncertainty facilitates the use of unimodal distributions in representing uncertainties in the models being implemented and can even enable use of linearized models in some examples.

As a further example, FIGS. 9A and 9B depict an enlarged view of the example catheter 502 which includes a plurality of coil sets 542, 544, and 546. The catheter actuation control (e.g., corresponding to control 522) is electrically connected with each of the coil sets for supplying electrical currents through one or more of the coils at each coil set to effect deflection of the catheter in a desired way in response to the magnetic field generated by the corresponding MRI system. In the example of FIG. 9A, each of the coil sets 542, 544, and 546 includes three orthogonal coils. In the cross-sectional view of FIG. 9A, the coil set 542 is shown to include one coil 552 wound circumferentially around or embedded in the catheter body. Another coil 554 includes coil windings disposed on diametrically opposed sides of the catheter body, such as in juxtaposition with respect to the coils 552. Another coil 554 can be disposed on diametrically opposed sides of the catheter body in juxtaposition with respect to the coils 552 and extending orthogonally with respect to the coils 554. Each of the coils 552, 554 and 556 has a number of turns wound around an axis, in which each of the respective axes is orthogonal to each other. In the particular orientation shown of FIG. 9A, the coil 554 includes windings around an axis that extends vertically through the catheter body, and the coil 556 includes windings around another axis that extends horizontally through the catheter body. Thus, the respective currents can be selectively applied to each coil to achieve desired deflection (e.g., steering) of the catheter 502 in response to the magnetic field provided by the associated MRI system 508. The use of multiple coils in the catheter 502 also mitigates actuation singularities that can occur in other designs. The multiple coil sets further enable controlled deflections of the catheter tips in three-dimensional space.

As a further example, to facilitate positioning the catheter tip via MRI actuated deflections (e.g., by energizing respective coils), the catheter body can be constructed from a material having a Young's modulus that is less than about 200 MPa (e.g., about 100 MPa or less). Examples of some materials include polyurethane, latex rubber and silicone rubber, each of which has a Young's modulus that is less than about 100 MPa, and in some cases a Young's modulus less than about 50 MPa to afford increased deflection performance. Other numbers and configurations of coils can be used in other examples.

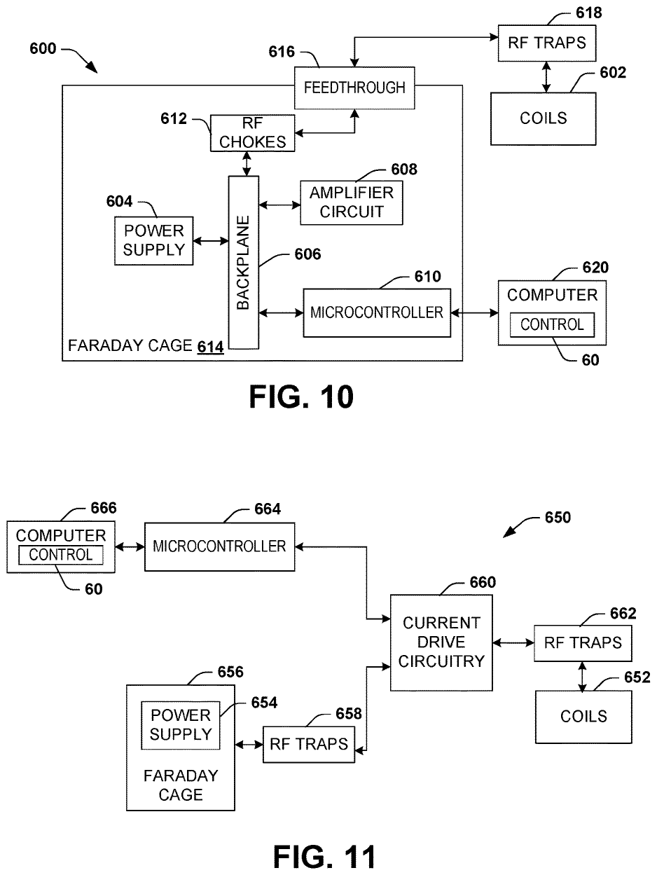

FIG. 10 is a block diagram showing an example of actuator control circuitry 600 that can be implemented for driving one or more actuator coils 602. The circuitry 600 can provide an example of the part of the intervention system 14 and coils on the device 18, as shown in FIG. 1. Accordingly, the description of FIG. 10 can also refer to certain aspects of the system 10 of FIG. 1.

In the example of FIG. 10, the circuitry 600 includes a power supply 604, a backplane 606, an amplifier circuit 608, a microcontroller 610, and one or more RF chokes 612. Such circuitry 604, 606, 60, 610, and 612 can be implemented within a Faraday cage 614, which can include a feedthrough (e.g., one or more feedthrough capacitors) 616 coupled to the coils through an arrangement of one or more RF traps 618. As described herein, the one or more actuator coils 602 are on a device, such as on or within a distal end portion of a catheter body, which are configured to deflect the catheter body in one or more directions based on current and magnetic field applied to the respective coils 602.

The backplane is configured to communicate data (e.g., instructions, feedback information and the like) and power among the circuitry residing within the Faraday cage 614. For example, the microcontroller 610 is configured to provide control data to the amplifier circuit 608 for driving the coils 602 based on instructions provided from a computer 620, which executes code to implement the control methods 60 (e.g., actuation control), as described herein. The computer 620 can be coupled to the microcontroller 610 through an optical link, such as an optical universal serial bus or other optical connection. The amplifier circuit 608 can include a number of transconductance (or other) amplifiers configured to amplify control signals provided by the microcontroller 610 to energize the one or more of the coils 602 based on the control methods 60. The RF chokes 612 can block high frequency current that can occur within an MR imaging space (e.g., space 16) where the circuitry is being used. A number of one or more RF traps (e.g., quadrupole ion traps) 618 can be coupled between the feedthrough circuit 616 and the one or more coils 602, which can depend on the distance of the coupling to the coils.

FIG. 11 is a block diagram showing another example of actuator control circuitry 650 that can be implemented for driving one or more actuator coils 652. The circuitry 650 can provide another example of part of the intervention system 14 and coils on the device 18, as described with respect to FIG. 1. Accordingly, the description of FIG. 11 can also refer to certain aspects of the description of FIG. 1.

In the example of FIG. 11, the circuitry 650 can be considered more distributed compared to the circuitry 600 of FIG. 10. The circuitry 650 includes a power supply 654 that is within a Faraday cage 656. The power supply 654 can include a switch mode power supply and distribution circuitry configured to distribute power (e.g., a DC voltage, such as about 12 V) to components of current drive circuitry 660 through one or more RF traps 658. The current drive circuitry 660 can include a number of driver circuits corresponding to the number of coils 652. That is, a separate driver can be provided for each coil, which is coupled to the respective coils 652 through one or more RF traps 662. The current drive circuit 660 thus is configured to supply current to each coil 652 responsive to power supplied at one or more power inputs of the drive circuitry and control signals supplied to one or more control inputs of the drive circuitry by a microcontroller 664. For example, the microcontroller 664 is configured to supply control instructions (e.g., data) to the control input(s) of the drive circuitry 660 as optical signals over an optical link (e.g., a fiber optic cable or other optical link) for driving the coils 652 based on instructions provided from a computer 666, which executes code to implement the control methods 60 (e.g., actuation control) as described herein. As described herein, the one or more actuator coils 652 are on a device, such as on or within a distal end portion of a catheter body, which are configured to deflect the catheter body in one or more directions based on current and magnetic field applied to the respective coils 602.

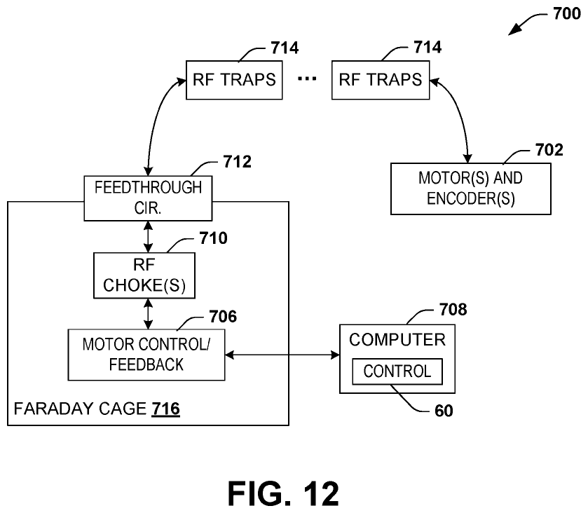

FIG. 12 is a block diagram showing an example of insertion control circuitry 700 that can be implemented for controlling insertion of a catheter and/or stiffening element by one or more motors or other actuators 702. As examples, the motors or other actuators 702 can be implemented as linear motors (e.g., a piezoelectric drive motor), rotary motors or a combination of linear and rotary motors. The circuitry 700 can provide another example of part of the intervention system 14 and the device 18, such as described with respect to FIG. 1. Accordingly, the description of FIG. 12 can also refer to certain aspects of the description of FIG. 1.

In an example, the one or more motors 702 can be coupled to the catheter body, directly or indirectly (e.g., through linkages and/or couplings), to implement axial movement of the catheter body. Also, or as alternative example, the one or more motors 702 can be coupled to a stiffening element that moves axially along a length of the catheter body, directly or indirectly (e.g., through linkages and/or couplings), to implement axial movement of the stiffening element. In example embodiments that include both a catheter and the stiffening element, a respective motor can be coupled to each of the catheter and stiffening element. The circuitry 700 thus can control the axial movement of the catheter body and stiffening element independently or in conjunction with each other according to application requirements or a given use environment. Each motor 702 can also include an associated encoder 704, which can be implemented as an absolute and/or relative encoder, configured to provide encoder data representative of motor position.

The circuitry 700 includes motor control/feedback circuitry 706 that can control the motors 702 based on feedback data and instructions from a computer 708. The computer 708 can be coupled to the motor control/feedback circuitry 706 through an optical link, such as described herein. As described herein, the computer 708 includes and/or implements insertion control methods 60. The feedback data can include information describing or derived from operation of the circuitry 700, such as voltages, current and/or motor position (e.g., based on encoder data from the encoder(s) 704). In some examples, the feedback data or instructions can also describe the condition and/or characterization of the image space, such as including localization data for the catheter tip, target location, force estimates and the like. In the example of FIG. 12, the motor control/feedback circuitry 706 is coupled to the motor(s) 702 through one or more RF chokes 710, feedthrough circuitry (capacitors) 712 and one or more RF traps 714. The RF choke(s) 710 and motor control/feedback circuitry 706 reside within a Faraday cage 716.

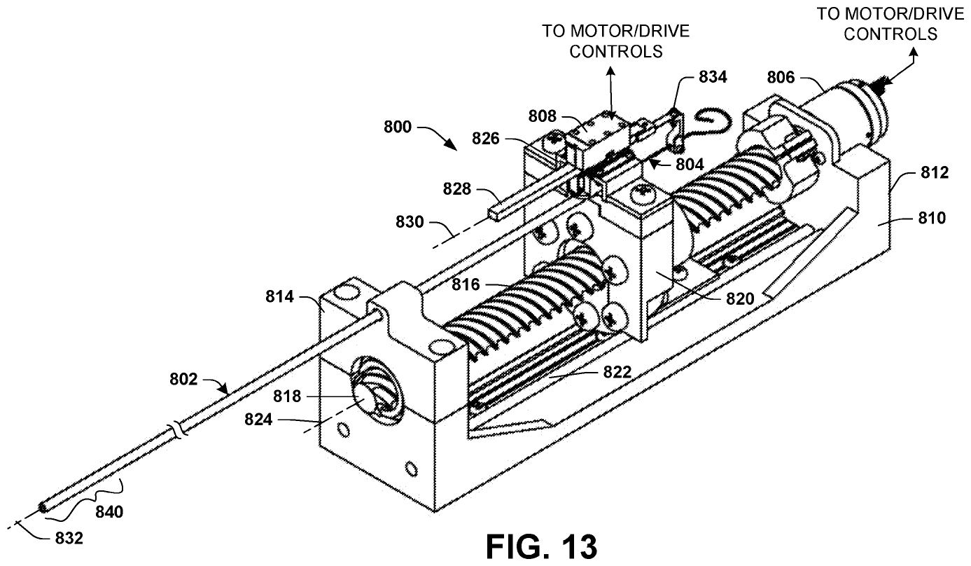

FIG. 13 is an example of an insertion apparatus 800 that can be implemented to axially position a catheter 802 and/or a stiffening element 804. In the example of FIG. 13, the apparatus 800 includes a catheter insertion motor 806, which is adapted to couple to the body of the catheter 802, and a stiffener motor 808, which is adapted to couple to the stiffening element. In some examples, the catheter insertion motor 806 is a rotary motor and the stiffener motor 808 is a linear motor. In other examples, different types of motors can be used. The motors 806 and 808 are examples of the motor(s) 702 of FIG. 12 and/or the intervention system 14 of FIG. 1, such that the description of FIG. 13 also refers to certain aspects of FIGS. 1 and 12. The apparatus 800 can be modified for use with various sizes and configurations of catheters and/or stiffening elements, such as might vary depending on the use environment and the intended target site.

By way of example, the apparatus includes a base 810 extending between proximal and distal ends 812 and 814, respectively. The catheter insertion motor 806 is mounted to a brace at the proximal end 812. For example, the catheter insertion motor 806 includes a shaft (not shown) coupled to a rotor of the motor, and the shaft is coupled to an elongated threaded rod (e.g., a screw or leadscrew) 816 that extends longitudinally from the motor shaft to terminate in a distal end 818 which is rotatably mounted (e.g., in a bushing) at the distal end 814 of the base 810. A first end of support bracket 820 can be mounted for movement along a guide rail 822 extending along a surface of the base 810 between the proximal and distal ends 812 and 814, and the threaded rod is spaced apart from (e.g., mounted above) the guide rail. The threaded rod 816 can be a leadscrew that rotates about a longitudinal central axis 824 extending through the rod and parallel to the guide rail 822.

The support bracket 820 can include a linkage (e.g., gear) along an interior passage that translates the rotation of the rod 816 into linear motion in a direction that is parallel to the axis 824. For example, the linkage of the support bracket 820 and rod 816 can define a rack and pinion or ball-screw actuator. The support bracket 820 can also include a fastening element to which a proximal end of the catheter 802 can be coupled to provide for axial movement of the catheter parallel to the axis 824 commensurate with linear movement of the support bracket 820. For example, the distal end of the base 810 can include an aperture through which a length of the catheter 802 (and objects inserted with a lumen of the catheter body) can traverse.

Another end of the support bracket 820, which is spaced apart from the guide rail, can include a respective motor mount 826 adapted to mount the motor 808 to the support bracket 820. The motor 808 can be a linear motor that includes an elongated drive member 828 that is moveable along an axis 830 of the linear motor responsive to actuation of the linear motor. The axis 830 is parallel to a longitudinal catheter axis 832 extending through the catheter body 802. The drive member 828 can form or be coupled to a force transmitting member of the motor 808. A proximal end of the drive member 828 can be coupled to a proximal portion of the stiffening element 804 through a support arm 836. The support arm thus is adapted to transfer linear motion (along the axis 830) from the driver member to the stiffening element 804. The stiffening element extends from the supported proximal portion to terminate in a distal end thereof that can be moved to an axial position relative to the catheter body.

As a further example, the stiffening element 804 is configured to be moveable axially along the length of the catheter body 802. In some examples, the stiffening element is sized and configured for axial movement within the lumen of the catheter. In other examples, the stiffening element 804 has a lumen extending longitudinally through the stiffening element and is configured for axial movement over the catheter body. The stiffening element is configured to provide mechanical support and inhibit bending of a portion the catheter body that is coextensive with the axial position of the stiffening element along the length of the catheter body and to permit bending of another part of the distal end portion of the catheter body that extends beyond the distal end of the stiffening element responsive to actuation of the at least one coil. In some examples, the stiffening element is configured to be rotated (e.g., manually-by hand-or responsive to actuation of another motor-not shown) relative to the catheter body 802 a central longitudinal axis of the stiffening element. The stiffening element 804 can be straight or curved, and depending on its shape can impart a corresponding shape to the more pliant catheter body in which the stiffening element is positioned.

Each of the motors 806 and 808 has respective inputs that are coupled to insertion control circuitry (e.g., instances of the insertion control circuitry 700) to receive control signals for actuating the respective motors. For example, actuation of the motor 806 is responsive to a first drive control signal (e.g., provided by device control 28 or motor control 706) and the actuation of the motor 808 is responsive to a second drive control signal (e.g., provided by device control 28 or motor control 706). Actuation of the motor 806 rotates the shaft 816, which causes corresponding linear movement of the support bracket and objects coupled to the support bracket, including the catheter 802, linear motor 808, and (in some situations) the stiffening element 804. Actuation of the motor 808 applies linear force to move the drive member 828 axially, which causes corresponding linear movement of the stiffening element 804. The motors 806 and 808 can thus be controlled to provide for independent or coordinated movement of the catheter 802 and stiffening element 804, as described herein.

The catheter 802 also includes a distal end portion 840, which can include one or more coils (e.g., coils 542, 544, 546, 602, or 652). Each of the coils at the distal end portion can be coupled to an actuation control circuitry by an arrangement of conductors and RF traps, such as described herein, in which the conductors can extend through one or more sidewall lumen extending through the catheter 802. The catheter coils can be selectively actuated based on actuation control methods (e.g., control methods 60 and actuation circuitry (e.g., actuation circuitry 524, 600, 650) to provide for deflection of the distal end portion in one or more directions. As described herein, the insertion control and coil actuation can be coordinated to facilitate moving the catheter along a desired trajectory, such as to a target site.

As a further example, FIGS. 14A, 14B and 14C is a conceptual diagram showing different amounts of deflection for a distal portion of a catheter 850, such as responsive to actuation of a respective catheter coil 852 while a stiffening element is at an axial position within the catheter lumen, as described herein. For example, FIG. 14A shows a distal portion of the catheter 850 having a first amount of deflection based on the coil 852 being actuated with the stiffening element 854 at a first axial position 856 within the catheter lumen. FIG. 14B shows a distal portion of the catheter 850 having a second amount of deflection based on the coil 852 being actuated with the stiffening element 854 at a second axial position 858. The second axial position is more distal from the first axial position resulting in a smaller bend radius (e.g., less deflection). FIG. 14C shows a distal portion of the catheter 850 having a third amount of deflection based on the coil 852 being actuated with the stiffening element 854 at a third axial position 860. The third axial position is more distal than the second axial position resulting in an even smaller bend radius.

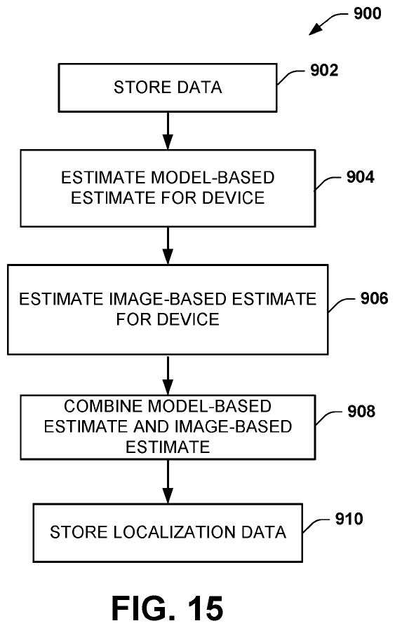

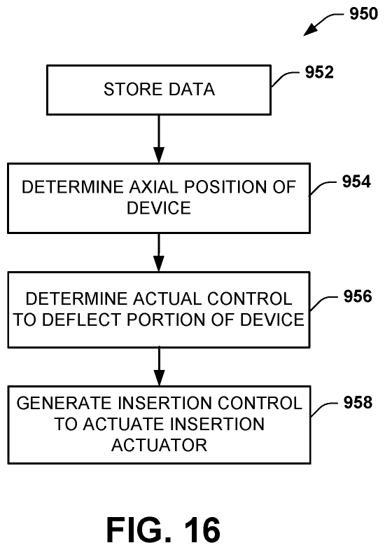

In view of the foregoing structural and functional features described above, example methods will be better appreciated with reference to FIGS. 15 and 16. While, for purposes of simplicity of explanation, the example methods are shown and described as executing serially, it is to be understood and appreciated that the examples are not limited by the illustrated order, as some actions could in other examples occur in different orders, multiple times and/or concurrently from that shown and described herein. The methods of FIGS. 16 and 17 can be implemented, in whole or in part, by systems and devices described herein, including the systems 10, the system 500 of FIG. 8, the circuits 600, 650, 700 and the devices 502 and 800.

FIG. 15 is a flow diagram of an example method 900 for localizing a device, such as a catheter. At 902, the method includes storing data (e.g., in memory, such as one or more non-transitory media). The data can include device model data representing a mathematical model of a device and anatomical data representing anatomy of a patient's body.

At 904, the method 900 includes estimating a model-based estimate of shape and/or location of the device based on the device model data, the anatomical data and/or actuation inputs applied to control current to at least one coil at the distal portion of the device. At 906, the method 900 includes estimating an image-based estimate of a shape and/or location of the device based on image artifacts detected in one or more images. The image artifacts are representative of magnetic actuation of the at least one coil in response to the actuation inputs. At 908, the method 900 includes combining the model-based estimate and the image-based estimate to provide localization data representative of the location, orientation and/or shape of the device relative to the anatomy.

In some examples, drive electronics are coupled to each one or more coils, and the method 900 includes controlling the drive electronics based on the localization data to apply current in a null-space of an actuation Jacobian for the device. The method 900 can also include determining an error between the model-based estimate and the image-based estimate, and determining a value representative of contact force at the distal end of the device based on the error. Also, or alternatively, the method 900 can also include determining an error between the model-based estimate and the image-based estimate, and determining a value representative of distributed forces along the body of the device based on the error.

Also, or as an alternative, the device includes at least one sensor at the distal end portion configured to provide a sensor value that varies depending on a shape of the distal end portion, and the method 900 includes estimating the shape of the distal end portion based on the sensor value, and controlling current applied to the at least one coil based on the estimated shape.

Also, or as an alternative, the device the device includes at least one sensor at the distal end portion configured to provide a sensor value that varies depending on a shape of the distal end portion, and the method 900 includes estimating the contact force at the distal end portion based on the sensor value, and controlling current applied to the at least one coil based on the estimated contact force.

Also, or as an alternative, the device the device is a catheter including a plurality of multi-axial coils disposed about a distal portion the catheter, in which each of the coils has respective axis positioned to provide for selective movement of the catheter relative to the respective axis, and the method 900 further comprises controlling current applied to each of the coils to selectively move one or more parts of the distal portion of the catheter based on the localization data.

FIG. 16 is a flow diagram of an example method 950 for coordinated actuation and insertion control. At 952, the method 900 includes storing data. The data include position data representative of a spatial position of at least a flexible distal portion of the device, and the device includes at least one actuation coil at a distal portion of the device. The data can also include target data representative of at least one of a location of a target site and a trajectory of the distal portion of the device to the target site.

At 954, the method 950 includes determining an axial position of a distal portion of a device based on the position data. At 956, the method includes determining an actuation control signal to provide current to the at least one coil to deflect the distal portion of the device based on the position data and the target data. At 958, the method includes generating an insertion control signal to actuate an insertion actuator coupled to the device to move at least the distal portion of the device axially, in which the insertion control signal and the actuation control signal are coordinated to advance the distal portion of the device along the trajectory.

In some examples, the device is a catheter that includes a plurality of multi-axial coils disposed about the catheter along the distal portion, in which each of the coils has a respective axis positioned to provide for selective movement of the catheter relative to the respective axis, and the method 950 further comprises controlling the insertion actuator to move at least the distal portion of the catheter axially in response to the insertion control signal. Also, or as an alternative, the catheter has a base at a proximal end thereof that is configured to rotate at least the distal portion of the catheter about a central axis extending therethrough.

In a further example, the catheter includes a lumen extending longitudinally through the catheter, and an elongated tool is configured to be inserted and move axially within the lumen of the catheter. The method 950 can include controlling the deflection of the catheter and the elongated tool inserted within the lumen based on magnetic actuation of the coils. Also, or as an alternative, the method 950 can include controlling the insertion actuator to axially move the catheter and the elongated tool inserted therein based on the insertion control signal.

Also, or as an alternative, the method 950 can include generating image data representing at least a portion of an image space, in which the image space includes at least one of the device or catheter and the target site, and controlling the at least one coil and the insertion actuator, cooperatively, based on the position data a relative location of the device or catheter and anatomy of the patient's body, in which the anatomy is determined based on the image data. The method can also include determining the relative location of the device or catheter based on a mathematical model describing a shape of the device or catheter shape and/or direct measurement from the image data.

In some examples, the imaging system is an MR imaging system and the image data is MR image data. The method 950 thus can also include controlling the MR imaging system to generate MR data based on RF pulses and magnetic field gradients provided within the image space during MR image acquisition, and reconstructing an image of image space based on the MR data.

The catheter devices, intervention systems and methods described herein can be particularly adapted to perform cardiac ablation interventions as well as perform brain/cerebral ablations, which can be implemented during interventional MRI of the tissue containing one or more ablation target sites. Other interventions than ablation can also utilize the systems and methods described herein. For example, percutaneous valves could be placed using a catheter which would automatically account for motion. Furthermore, the systems and methods herein can be extended to employ external beam radiation techniques to multiple different areas of the body. This general approach could be used to automatically guide the external beam for ablation purposes.

### EXAMPLE EMBODIMENTS

Several aspects of the present technology are set forth in the following numbered examples.
1. A system, comprising:
   one or more non-transitory media storing data and executable instructions, in which the data comprises position data representative of a spatial position of at least a flexible distal portion of a device, and target data representative of at least one of a location of a target site and a trajectory of the distal portion of the device to the target site; and
   a processor configured to access the non-transitory media and perform a method comprising:
      determining an axial position of the distal portion of the device based on the position data;
      determining an actuation control signal to provide current to at least one coil at the distal portion of the device to deflect the distal portion of the device based on the position data and the target data; and
      determining an insertion control signal to actuate an insertion actuator coupled to the device to move at least the distal portion of the device axially, in which the insertion control signal and the actuation control signal are coordinated to advance the distal portion of the device along the trajectory.
2. The system of example 1, wherein the device is a catheter, and the system further comprises:
   the catheter, in which the catheter includes a plurality of multi-axial coils disposed about the catheter, and each of the coils has a respective axis positioned to provide for selective movement of the catheter relative to the respective axis; and
   the insertion actuator, in which the insertion actuator is configured to move at least the distal portion of the catheter axially in response to the insertion control signal.
3. The system of example 2, wherein the catheter has a base at a proximal end thereof that is configured to rotate at least the distal portion of the catheter about a central axis extending therethrough.
4. The system according to example 2 or 3, further comprising:
   an elongated tool, wherein the catheter includes a lumen extending longitudinally through the catheter, the elongated tool configured to be inserted and move axially within the lumen of the catheter,
   wherein the processor is configured to at least one of:
      implement magnetic actuation of at least one of the coils to control deflection of the catheter and the elongated tool inserted within the lumen; and
      implement insertion control to control axial insertion of the catheter and the elongated tool inserted therein.
5. The system of example 1, wherein the system further comprises:
   the device, in which the device is a catheter having an elongate catheter body extending between a proximal end portion and a distal end portion, the catheter body has a lumen extending longitudinally through the catheter body, the distal end portion is flexible, and the at least one coil is disposed at the distal end portion of the catheter body and configured to deflect the distal end portion in response to a magnetic field.
6. The system of example 5, further comprising:
   an elongated stiffening element extending between spaced apart proximal and distal ends thereof, in which the stiffening element is configured to be moveable axially along the length of the catheter body.
7. The system of example 6, wherein:
   the stiffening element is sized and configured for axial movement within the lumen of the catheter, or
   the stiffening element has a lumen extending longitudinally through the stiffening element and is configured for axial movement over the catheter body.
8. The system according to example 6 or 7, wherein the stiffening element is configured to provide mechanical support and inhibit bending of a portion the catheter body that is coextensive with an axial position of the stiffening element along the length of the catheter body and to permit bending of another part of the distal end portion of the catheter body that extends beyond the distal end of the stiffening element responsive to actuation of the at least one coil.
9. The system according to any one of examples 6, 7, or 8, wherein the stiffening element is configured to be rotated relative to the catheter body about a central longitudinal axis extending through the stiffening element.
10. The system according to any one of examples 6, 7, 8, or 9, wherein the stiffening element is straight or curved.
11. The system according to example 9 or 10, wherein the catheter is configured to have a shape based on at least one of an axial position of the stiffening element and the catheter body, an amount of the rotation of the stiffening element relative to the catheter body, and magnetic actuation of the at least one coil.
12. The system according to any one of examples 5, 6, 7, 8, 9, 10 or 11, wherein the at least one coil includes a plurality of coils disposed about the distal end portion of the elongated body of the catheter.
13. The system according to any one of examples 6, 7, 8, 9, 10 or 11, further comprising an actuation controller configured to control actuation of each coil to deflect the distal end portion based on the actuation control signal, in which the position of the stiffening element within the lumen during the actuation is operative to control an amount that the distal end portion deflects.
14. The system according to any one of examples 1, 5, 6, or 7, wherein the data further includes device model data representing a model of the device and anatomical data representing anatomy of a patient's body, and the method, which is executed by the processor, further comprises:
   estimating a model-based estimate of shape and/or location of the device based on the device model data, the anatomical data and actuation inputs applied to control current to the at least one coil at the distal portion of the device;
   estimating an image-based estimate of a shape and/or location of the device based on image artifacts detected in one or more images, in which the image artifacts are representative of magnetic actuation of the at least one coil in response to the actuation inputs; and
   combining the model-based estimate and the image-based estimate to provide localization data representative of the location, orientation and/or shape of the device.
15. The system of example 14, further comprising:
   drive electronics coupled to the at least one coil; and
   motion control circuitry configured to control the drive electronics to apply current in a null-space of an actuation Jacobian for the device.
16. The system according to example 14 or 15, wherein the processor is further configured to:
   determine an error between the model-based estimate and the image-based estimate; and
   determine a value representative of contact force at the distal end of the device based on the error.
17. The system according to any one of examples 14, 15, or 16, wherein the processor is further configured to:
   determine an error between the model-based estimate and the image-based estimate; and
   determined a value representative of distributed forces along the body of the device based on the error.
18. The system according to any one of examples 14, 15, 16, or 17, wherein the device includes at least one sensor at the distal end portion configured to provide a sensor value that varies depending on a shape of the distal end portion, wherein the processor is further configured to estimate the shape of the distal end portion based on the sensor value.
19. The system according to any one of examples 14, 15, 16, or 17, wherein the device includes at least one sensor at the distal end portion configured to provide a sensor value that varies depending on a shape of the distal end portion, wherein the processor is further configured to estimate the contact force at the distal end portion based on the sensor value.
20. The system according to any one of examples 14, 15, 16, 17, 18, or 19, wherein the system comprises the device and the device is a catheter, in which the catheter includes a plurality of multi-axial coils disposed about the catheter, in which each of the coils has respective axis positioned to provide for selective movement of the catheter relative to the respective axis
21. The system according to any preceding example, further comprising:
   an imaging system to provide image data representing at least a portion of an image space, in which the image space includes at least one of the device or catheter and the target site within a patient's body;
   wherein the processor is further configured to control the at least one coil and the insertion actuator based on a relative location of the device or catheter and anatomy of the patient's body, in which the anatomy is determined based on the image data.
22. The system of example 21, wherein the processor is further configured to determine the relative location based on a mathematical model describing a shape of the device or catheter and/or direct measurement from the image data.
23. The system according to example 21 or 22, wherein the imaging system comprises a magnetic resonance (MR) imaging system and the image data is MR image data, the MR imaging system is configured to generate MR data based on RF pulses and magnetic field gradients provided within the image space during MR image acquisition, and the processor is further programmed to execute instructions to reconstruct an image of image space based on the MR data.
24. A method, comprising:
   determining an axial position of a distal portion of a device based on position data, in which the position data is representative of a spatial position of at least a flexible distal portion of the device, and the device includes at least one actuation coil at a distal portion of the device;
      determining an actuation control signal to provide current to the at least one coil to deflect the distal portion of the device based on the position data and target data, in which the target data is representative of at least one of a location of a target site and a trajectory of the distal portion of the device to the target site; and
      generating an insertion control signal to actuate an insertion actuator coupled to the device to move at least the distal portion of the device axially, in which the insertion control signal and the actuation control signal are coordinated to advance the distal portion of the device along the trajectory.
25. The method of example 24, wherein the device is a catheter that includes a plurality of multi-axial coils disposed about the catheter along the distal portion, in which each of the coils has a respective axis positioned to provide for selective movement of the catheter relative to the respective axis, and the method further comprises controlling the insertion actuator to move at least the distal portion of the catheter axially in response to the insertion control signal.
26. The method of example 25, wherein the catheter has a base at a proximal end thereof that is configured to rotate at least the distal portion of the catheter about a central axis extending therethrough.
27. The method according to example 25 or 26, wherein the catheter includes a lumen extending longitudinally through the catheter, and an elongated tool is configured to be inserted and move axially within the lumen of the catheter, and the method further comprises at least one of:
   controlling the deflection of the catheter and the elongated tool inserted within the lumen based on magnetic actuation of the coils; and
   controlling the insertion actuator to axially move the catheter and the elongated tool inserted therein based on the insertion control signal.
28. The method according to any one of examples 24, 25, 26, or 27, further comprising:
   generating image data representing at least a portion of an image space, in which the image space includes at least one of the device or catheter and the target site;
   controlling the at least one coil and the insertion actuator, cooperatively, based on the position data a relative location of the device or catheter and anatomy of the patient's body, in which the anatomy is determined based on the image data.
29. The method of example 28, further comprising determining the relative location of the device or catheter based on a mathematical model describing a shape of the device or catheter shape and/or direct measurement from the image data.
30. The method according to example 28 or 29, wherein the imaging system comprises a magnetic resonance (MR) imaging system and the image data is MR image data, and the method further comprises:
   controlling the MR imaging system to generate MR data based on RF pulses and magnetic field gradients provided within the image space during MR image acquisition, and
   reconstructing an image of image space based on the MR data.
31. The method of example 24, wherein the device is a catheter having an elongate catheter body extending between a proximal end portion and a distal end portion, the catheter body has a lumen extending longitudinally through the catheter body, the distal end portion is flexible, the at least one coil is disposed at the distal end portion of the catheter body and configured to deflect the distal end portion in response to a magnetic field.
32. The method of example 31, further comprising:
   moving an elongated stiffening element, extending between spaced apart proximal and distal ends thereof, axially along the length of the catheter body.
33. The method of example 32, wherein the stiffening element is sized and configured for axial movement within the lumen of the catheter, or
   the stiffening element has a lumen extending longitudinally through the stiffening element and is configured for axial movement over the catheter body, and
   wherein the method further comprises moving the stiffening element axially relative to the catheter body.
34. The method according to example 32 or 33, wherein the stiffening element is configured to provide mechanical support and inhibit bending of a portion the catheter body that is coextensive with the axial position of the stiffening element along the length of the catheter body, and
   wherein the method further comprises deflecting another part of the distal end portion of the catheter body that extends beyond the distal end of the stiffening element responsive to actuation of the at least one coil.
35. The method according to any one of examples 32, 33, or 34, further comprising rotating the stiffening element relative to the catheter body about a central longitudinal axis extending through the stiffening element.
36. The method according to any one of examples 32, 33, 34, or 35 wherein the stiffening element is straight or curved.
37. The method according to example 35 or 36, further comprising moving the stiffening element axially to an axial position along the catheter body, wherein the catheter has a shape based on at least one of the axial position of the stiffening element along the catheter body, an amount of the rotation of the stiffening element relative to the catheter body, and magnetic actuation of the at least one coil.
38. The method according to any one of examples 31, 32, 33, 34, 35, 36, or 37, wherein the at least one coil includes a plurality of coils disposed about the distal end portion of the elongated body of the catheter.
39. The method of example 38, further comprising controlling actuation of each coil to deflect the distal end portion based on the actuation control signal, in which the position of the stiffening element within the lumen during the actuation is operative to control an amount that the distal end portion deflects.
40. The method according to any one of examples 24, 31, 32, or 33, wherein the data further includes device model data representing a model of the device and anatomical data representing anatomy of a patient's body, and the method further comprises:
   estimating a model-based estimate of shape and/or location of the device based on the device model data, the anatomical data and actuation inputs applied to control current to the at least one coil at the distal portion of the device;
   estimating an image-based estimate of a shape and/or location of the device based on image artifacts detected in one or more images, in which the image artifacts are representative of magnetic actuation of the at least one coil in response to the actuation inputs; and
   combining the model-based estimate and the image-based estimate to provide localization data representative of the location, orientation and/or shape of the device.
41. The method of example 40, further comprising:
   controlling drive electronics, which are coupled to the at least one coil, to apply current in a null-space of an actuation Jacobian for the device.
42. The method according to example 40 or 41, further comprising:
   determining an error between the model-based estimate and the image-based estimate; and
   determining a value representative of contact force at the distal end of the device based on the error.
43. The method according to any one of examples 40, 41, or 42, further comprising:
   determining an error between the model-based estimate and the image-based estimate; and
   determining a value representative of distributed forces along the body of the device based on the error.
44. The method according to any one of examples 40, 41, 42, or 43, further comprising:
   providing a sensor signal having a sensor value that varies depending on a shape of the distal end portion; and
   estimating the shape of the distal end portion based on the sensor value.
45. The method according to any one of examples 40, 41, 42, or 43, further comprising:
   providing a sensor signal a sensor value that varies depending on a shape of the distal end portion; and
   estimating the contact force at the distal end portion based on the sensor value.
46. The method according to any one of examples 40, 41, 42, 43, 44, or 45, wherein the device is a catheter including a plurality of multi-axial coils disposed about the catheter, in which each of the coils selectively moves a respective portion of the catheter relative to the respective axis responsive to the actuation signal.
47. A system, comprising:
   one or more non-transitory media storing data and executable instructions, in which the data comprises device model data representing a mathematical model of a device and anatomical data representing anatomy of a patient's body; and
   a processor configured to access the memory and perform a method comprising:
      estimating a model-based estimate of shape and/or location of the device based on the device model data, the anatomical data and actuation inputs applied to control current to at least one coil at the distal portion of the device;
      estimating an image-based estimate of a shape and/or location of the device based on image artifacts detected in one or more images, in which the image artifacts are representative of magnetic actuation of the at least one coil in response to the actuation inputs; and
      combining the model-based estimate and the image-based estimate to provide localization data representative of the location, orientation and/or shape of the device relative to the anatomy.
48. The system of example 47, further comprising:
   drive electronics coupled to the at least one coil; and
   motion control circuitry configured to control the drive electronics based on the localization data to apply current in a null-space of an actuation Jacobian for the device.
49. The system according to example 47 or 48, wherein the processor is further configured to:
   determine an error between the model-based estimate and the image-based estimate; and
   determine a value representative of contact force at the distal end of the device based on the error.
50. The system according to any one of examples 47, 48, or 49, wherein the processor is further configured to:
   determine an error between the model-based estimate and the image-based estimate; and
   determine a value representative of distributed forces along the body of the device based on the error.
51. The system according to any one of examples 47, 48, 49, or 50, wherein the device includes at least one sensor at the distal end portion configured to provide a sensor value that varies depending on a shape of the distal end portion, wherein the processor is further configured to estimate the shape of the distal end portion based on the sensor value.
52. The system according to any one of examples 47, 48, 49, or 50, wherein the device includes at least one sensor at the distal end portion configured to provide a sensor value that varies depending on a shape of the distal end portion, wherein the processor is further configured to estimate the contact force at the distal end portion based on the sensor value.
53. The system according to any one of examples 47, 48, 49, 50, 51 or 52, wherein the system comprises the device and the device is a catheter, in which the catheter includes a plurality of multi-axial coils disposed about the catheter, in which each of the coils has respective axis positioned to provide for selective movement of the catheter relative to the respective axis.
54. A method comprising:
   storing, in one or more non-transitory media, device model data representing a mathematical model of a device and anatomical data representing anatomy of a patient's body;
   estimating a model-based estimate of shape and/or location of the device based on the device model data, the anatomical data and actuation inputs applied to control current to at least one coil at the distal portion of the device;
   estimating an image-based estimate of a shape and/or location of the device based on image artifacts detected in one or more images, in which the image artifacts are representative of magnetic actuation of the at least one coil in response to the actuation inputs; and
   combining the model-based estimate and the image-based estimate to provide localization data representative of the location, orientation and/or shape of the device relative to the anatomy.
55. The method of example 54, wherein drive electronics are coupled to the at least one coil, and the method includes controlling the drive electronics based on the localization data to apply current in a null-space of an actuation Jacobian for the device.
56. The method according to example 54 or 55, further comprising
   determining an error between the model-based estimate and the image-based estimate; and
   determining a value representative of contact force at the distal end of the device based on the error.
57. The method according to any one of examples 54, 55, or 56, further comprising:
   determining an error between the model-based estimate and the image-based estimate; and
   determining a value representative of distributed forces along the body of the device based on the error.
58. The method according to any one of examples 54, 55, 56, or 57, wherein the device includes at least one sensor at the distal end portion configured to provide a sensor value that varies depending on a shape of the distal end portion, and the method further comprises:
   estimating the shape of the distal end portion based on the sensor value; and
   controlling current applied to the at least one coil based on the estimated shape.
59. The method according to any one of examples 54, 55, 56, or 57, wherein the device includes at least one sensor at the distal end portion configured to provide a sensor value that varies depending on a shape of the distal end portion, and the method further comprises:
   estimating the contact force at the distal end portion based on the sensor value; and
   controlling current applied to the at least one coil based on the estimated contact force.
60. The method according to any one of examples 54, 55, 56, 57, 58, or 59, wherein the device is a catheter including a plurality of multi-axial coils disposed about a distal portion the catheter, in which each of the coils has respective axis positioned to provide for selective movement of the catheter relative to the respective axis, and the method further comprises controlling current applied to each of the coils to selectively move one or more parts of the distal portion of the catheter based on the localization data.
61. The method according to any one of examples 54, 55, 56, 57, 58, or 59, wherein the device is a catheter comprising:
   an elongate catheter body extending between a proximal end portion and a distal end portion, in which the catheter body has a lumen extending longitudinally through the catheter body, and the distal end portion is flexible, the at least one coil is disposed at the distal end portion of the catheter body and configured to deflect the distal end portion in response to a magnetic field; and
   an elongated stiffening element extending between spaced apart proximal and distal ends thereof, in which the stiffening element is configured to be moveable axially along the length of the catheter body, and
   wherein the method further comprises controlling the magnetic field to deflect the distal end portion by an amount that also depends on an axial position of the stiffening element relative to the catheter body.
62. The method of example 61, further comprising moving the stiffening element axially along the length of the catheter body to the axial position.
63. The method of example 62, wherein the stiffening element is sized and configured for axial movement within the lumen of the catheter, or
   the stiffening element has a lumen extending longitudinally through the stiffening element and is configured for axial movement over the catheter body, and
   wherein the method further comprises moving the stiffening element axially within the lumen or over the catheter body to the axial position.
64. The method according to example 62 or 63, wherein the stiffening element is configured to provide mechanical support and inhibit bending of a portion the catheter body that is coextensive with the axial position of the stiffening element along the length of the catheter body, and
   wherein the method further comprises deflecting another part of the distal end portion of the catheter body that extends beyond the distal end of the stiffening element responsive to actuation of the at least one coil.
65. The method according to any one of examples 62, 63, or 64, wherein the stiffening element is straight or curved.
66. The method according to any one of examples 62, 63, 64, or 65, further comprising rotating the stiffening element relative to the catheter body about a central longitudinal axis extending through the stiffening element.
67. The method according to example 65 or 66, further comprising moving the stiffening element axially to the axial position along the catheter body, wherein the catheter has a shape based on at least one of the axial position of the stiffening element along the catheter body, an amount of the rotation of the stiffening element relative to the catheter body, and magnetic actuation of the at least one coil.
68. The method according to any one of examples 61, 62, 63, 64, 65, 66, or 67, wherein the at least one coil includes a plurality of coils disposed about the distal end portion of the elongated body of the catheter.
69. The method of example 68, further comprising controlling actuation of each coil to deflect the distal end portion based on the actuation control signal, in which the position of the stiffening element within the lumen during the actuation is operative to control an amount that the distal end portion deflects.
70. A catheter comprising:
   an elongate catheter body extending between a proximal end portion and a distal end portion, in which the catheter body has a lumen extending longitudinally through the catheter body, and the distal end portion is flexible;
   a coil disposed at the distal end portion of the catheter body and configured to deflect the distal end portion in response to a magnetic field; and
   an elongated stiffening element extending between spaced apart proximal and distal ends thereof, in which the stiffening element is configured to be moveable axially along the length of the catheter body.
71. The catheter of example 70, wherein:
   the stiffening element is sized and configured for axial movement within the lumen of the catheter, or
   the stiffening element has a lumen extending longitudinally through the stiffening element and is configured for axial movement over the catheter body.
72. The catheter according to example 70 or 71, wherein the stiffening element is configured to provide mechanical support and inhibit bending of a portion the catheter body that is coextensive with the axial position of the stiffening element along the length of the catheter body and to permit bending of another part of the distal end portion of the catheter body that extends beyond the distal end of the stiffening element responsive to actuation of the coil.
73. The catheter according to any of examples 70, 71, or 72, wherein the stiffening element is configured to be rotated relative to the catheter body about a central longitudinal axis extending through the stiffening element.
74. The catheter according to any of examples 70, 71, 72, or 73, wherein the stiffening element is straight or curved.
75. The catheter according to example 73 or 74, wherein the catheter is configured to have a shape based on an axial position of the stiffening element and the catheter body, an amount of the rotation of the stiffening element relative to the catheter body, and magnetic actuation of the coil.
76. The catheter according to any of examples 70, 71, 72, 73, 74, or 75, wherein the coil includes a plurality of coils disposed about the distal end portion of the elongated body of the catheter.
77. The catheter of any of examples 70, 71, 72, 73, 74, 75, or 76, further comprising an actuation controller configured to control actuation of each coil to deflect the distal end portion, in which the position of the stiffening element within the lumen during the actuation is operative to control an amount that the distal end portion deflects.

In view of the foregoing structural and functional description, those skilled in the art will appreciate that portions of the invention may be embodied as a method, data processing system, or computer program product. Accordingly, these portions of the invention may take the form of an entirely hardware embodiment, an entirely software embodiment, or an embodiment combining software and hardware. Furthermore, portions of the invention may be a computer program product on a computer-usable storage medium having computer readable program code on the medium. Any suitable computer-readable medium may be utilized including, but not limited to, static and dynamic storage devices, hard disks, optical storage devices, and magnetic storage devices.

Certain embodiments of the invention have also been described herein with reference to block illustrations of methods, systems, and computer program products. It will be understood that blocks of the illustrations, and combinations of blocks in the illustrations, can be implemented by computer-executable instructions. These computer-executable instructions may be provided to one or more processor of a general purpose computer, special purpose computer (e.g., as part of the imaging system), or other programmable data processing apparatus (or a combination of devices and circuits) to produce a machine, such that the instructions, which execute via the processor, implement the functions specified in the block or blocks.

These computer-executable instructions may also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

All references, publications, and patents cited in the present application are herein incorporated by reference in their entirety.

1. A system, comprising:
one or more non-transitory media storing data and executable instructions, in which the data comprises position data representative of a spatial position of at least a flexible distal portion of a device, and target data representative of at least one of a location of a target site and a trajectory of the distal portion of the device to the target site; and
a processor configured to access the non-transitory media and perform a method comprising:
determining an axial position of the distal portion of the device based on the position data;
determining an actuation control signal to provide current to at least one coil at the distal portion of the device to deflect the distal portion of the device based on the position data and the target data; and
determining an insertion control signal to actuate an insertion actuator coupled to the device to move at least the distal portion of the device axially, in which the insertion control signal and the actuation control signal are coordinated to advance the distal portion of the device along the trajectory.

2. The system of claim 1, wherein the device is a catheter, and the system further comprises:
the catheter, in which the catheter includes a plurality of multi-axial coils disposed about the catheter, and each of the coils has a respective axis positioned to provide for selective movement of the catheter relative to the respective axis; and
the insertion actuator, in which the insertion actuator is configured to move at least the distal portion of the catheter axially in response to the insertion control signal.

3. The system of claim 2, wherein the catheter has a base at a proximal end thereof that is configured to rotate at least the distal portion of the catheter about a central axis extending therethrough.

4. The system of claim 2, further comprising:
an elongated tool, wherein the catheter includes a lumen extending longitudinally through the catheter, the elongated tool configured to be inserted and move axially within the lumen of the catheter,
wherein the processor is configured to at least one of:
implement magnetic actuation of at least one of the coils to control deflection of the catheter and the elongated tool inserted within the lumen; and
implement insertion control to control axial insertion of the catheter and the elongated tool inserted therein.

5. The system of claim 1, wherein the system further comprises:
the device, in which the device is a catheter having an elongate catheter body extending between a proximal end portion and a distal end portion, the catheter body has a lumen extending longitudinally through the catheter body, the distal end portion is flexible, and the at least one coil is disposed at the distal end portion of the catheter body and configured to deflect the distal end portion in response to a magnetic field; and
an elongated stiffening element extending between spaced apart proximal and distal ends thereof, in which the stiffening element is configured to be moveable axially along the length of the catheter body.

6. (canceled)

7. The system of claim 5, wherein:
the stiffening element is sized and configured for axial movement within the lumen of the catheter, or
the stiffening element has a lumen extending longitudinally through the stiffening element and is configured for axial movement over the catheter body.

8. The system of claim 5, wherein the stiffening element is configured to provide mechanical support and inhibit bending of a portion the catheter body that is coextensive with an axial position of the stiffening element along the length of the catheter body and to permit bending of another part of the distal end portion of the catheter body that extends beyond the distal end of the stiffening element responsive to actuation of the at least one coil.

9. The system of claim 5, wherein the stiffening element is configured to be rotated relative to the catheter body about a central longitudinal axis extending through the stiffening element, wherein the catheter is configured to have a shape based on at least one two of an axial position of the stiffening element and the catheter body, an amount of the rotation of the stiffening element relative to the catheter body, and magnetic actuation of the at least one coil.

10-13. (canceled)

14. The system of claim 1, wherein the data further includes device model data representing a model of the device and anatomical data representing anatomy of a patient's body, and the method, which is executed by the processor, further comprises:
estimating a model-based estimate of shape and/or location of the device based on the device model data, the anatomical data and actuation inputs applied to control current to the at least one coil at the distal portion of the device;
estimating an image-based estimate of a shape and/or location of the device based on image artifacts detected in one or more images, in which the image artifacts are representative of magnetic actuation of the at least one coil in response to the actuation inputs; and
combining the model-based estimate and the image-based estimate to provide localization data representative of the location, orientation and/or shape of the device.

15. The system of claim 14, further comprising:
drive electronics coupled to the at least one coil; and
motion control circuitry configured to control the drive electronics to apply current in a null-space of an actuation Jacobian for the device.

16. The system of claim 14, wherein the processor is further configured to:
determine an error between the model-based estimate and the image-based estimate; and
determine a value representative of at least one of contact force at the distal end of the device and distributed forces along the body of the device based on the error.

17. (canceled)

18. The system of claim 14, wherein the device includes at least one sensor at the distal end portion configured to provide a sensor value that varies depending on a shape of the distal end portion, wherein the processor is further configured to estimate at least one of the shape of the distal end portion and the contact force at the distal end portion based on the sensor value.

19. (canceled)

20. (canceled)

21. The system of claim 1, further comprising:
an imaging system to provide image data representing at least a portion of an image space, in which the image space includes at least one of the device or catheter and the target site within a patient's body, wherein the imaging system comprises a magnetic resonance (MR) imaging system and the image data is MR image data, the MR imaging system is configured to generate MR data based on RF pulses and magnetic field gradients provided within the image space during MR image acquisition, and the processor is further programmed to execute instructions to reconstruct an image of image space based on the MR data, and
wherein the processor is further configured to control the at least one coil and the insertion actuator based on a relative location of the device or catheter and anatomy of the patient's body, in which the anatomy is determined based on the image data, wherein the processor is further configured to determine the relative location based on a mathematical model describing a shape of the device or catheter and/or direct measurement from the image data.

22-23. (canceled)

24. A method, comprising:
determining an axial position of a distal portion of a device based on position data, in which the position data is representative of a spatial position of at least a flexible distal portion of the device, and the device includes at least one actuation coil at a distal portion of the device;
determining an actuation control signal to provide current to the at least one coil to deflect the distal portion of the device based on the position data and target data, in which the target data is representative of at least one of a location of a target site and a trajectory of the distal portion of the device to the target site; and
generating an insertion control signal to actuate an insertion actuator coupled to the device to move at least the distal portion of the device axially, in which the insertion control signal and the actuation control signal are coordinated to advance the distal portion of the device along the trajectory.

25. The method of claim 24, wherein the device is a catheter that includes a plurality of multi-axial coils disposed about the catheter along the distal portion, in which each of the coils has a respective axis positioned to provide for selective movement of the catheter relative to the respective axis, and the method further comprises controlling the insertion actuator to move at least the distal portion of the catheter axially in response to the insertion control signal.

26. (canceled)

27. The method of claim 25, wherein the catheter includes a lumen extending longitudinally through the catheter, and an elongated tool is configured to be inserted and move axially within the lumen of the catheter, and the method further comprises at least one of:
controlling the deflection of the catheter and the elongated tool inserted within the lumen based on magnetic actuation of the coils; and
controlling the insertion actuator to axially move the catheter and the elongated tool inserted therein based on the insertion control signal.

28. The method of claim 25, further comprising:
generating image data representing at least a portion of an image space, in which the image space includes at least one of the device or catheter and the target site;
controlling the at least one coil and the insertion actuator, cooperatively, based on the position data a relative location of the device or catheter and anatomy of the patient's body, in which the anatomy is determined based on the image data; and
determining the relative location of the device or catheter based on a mathematical model describing a shape of the device or catheter shape and/or direct measurement from the image data.

29. (canceled)

30. The method of claim 28, wherein the imaging system comprises a magnetic resonance (MR) imaging system and the image data is MR image data, and the method further comprises:
controlling the MR imaging system to generate MR data based on RF pulses and magnetic field gradients provided within the image space during MR image acquisition, and
reconstructing an image of image space based on the MR data.

31. The method of claim 24, wherein the device is a catheter having an elongate catheter body extending between a proximal end portion and a distal end portion, the catheter body has a lumen extending longitudinally through the catheter body, the distal end portion is flexible, the at least one coil is disposed at the distal end portion of the catheter body and configured to deflect the distal end portion in response to a magnetic field.

32. The method of claim 31, further comprising:
moving an elongated stiffening element, extending between spaced apart proximal and distal ends thereof, axially along the length of the catheter body, wherein the stiffening element is sized and configured for axial movement within the lumen of the catheter, or
the stiffening element has a lumen extending longitudinally through the stiffening element and is configured for axial movement over the catheter body, and
wherein the method further comprises moving the stiffening element axially relative to the catheter body,
wherein the stiffening element is configured to provide mechanical support and inhibit bending of a portion the catheter body that is coextensive with an axial position of the stiffening element along the length of the catheter body, and
wherein the method further comprises deflecting another part of the distal end portion of the catheter body that extends beyond the distal end of the stiffening element responsive to actuation of the at least one coil.

33-39. (canceled)

40. The method of claim 24, wherein the data further includes device model data representing a model of the device and anatomical data representing anatomy of a patient's body, and the method further comprises:
estimating a model-based estimate of shape and/or location of the device based on the device model data, the anatomical data and actuation inputs applied to control current to the at least one coil at the distal portion of the device;
estimating an image-based estimate of a shape and/or location of the device based on image artifacts detected in one or more images, in which the image artifacts are representative of magnetic actuation of the at least one coil in response to the actuation inputs; and
combining the model-based estimate and the image-based estimate to provide localization data representative of the location, orientation and/or shape of the device.

41. The method of claim 40, further comprising:
controlling drive electronics, which are coupled to the at least one coil, to apply current in a null-space of an actuation Jacobian for the device;
determining an error between the model-based estimate and the image-based estimate; and
determining at least one of a value representative of contact force at the distal end of the device based on the error and a value representative of distributed forces along the body of the device based on the error.

42-77. (canceled)
